(19) United States
(12) Patent Application Publication
(10) Pub. No.: US 2022/0249596 A1
(43) Pub. Date: Aug. 11, 2022

(54) PIC1 PEPTIDE COMPOSITIONS AND METHODS OF USE THEREOF FOR TREATMENT OF HYPOXIC ISCHEMIC ENCEPHALOPATHY

(71) Applicant: REALTA HOLDINGS, LLC, Norfolk, VA (US)

(72) Inventors: Neel K. KRISHNA, Norfolk, VA (US); Kenji CUNNION, Norfolk, VA (US); Tushar SHAH, Norfolk, VA (US)

(73) Assignee: REALTA HOLDINGS, LLC, Norfolk, VA (US)

(21) Appl. No.: 17/613,527

(22) PCT Filed: May 28, 2020
(86) PCT No.: PCT/US2020/034789

§ 371 Date: Nov. 23, 2021

Related U.S. Application Data
(60) Provisional application No. 62/853,377, filed on May 28, 2019.

(51) Int. Cl. A61K 38/10; A61K 47/60; A61P 9/10

(52) U.S. Cl. CPC A61K 38/10; A61P 9/10; A61K 47/60

(57) ABSTRACT

A method of treating hypoxic ischemic encephalopathy (HIE) is described in which a classical complement pathway inhibitor is administered to a neonate. The classical pathway inhibitor can be PIC1, and may have one or more PEG moieties. Treatment of the neonate with the classical complement pathway inhibitor can inhibit inflammation and oxidative damage, such as might arise from ischemia-reperfusion injury. Improvement in fine motor performance, spatial memory and may be achieved. The method may be effective to treat or prevent cerebral palsy.

### CROSS-REFERENCE TO RELATED APPLICATIONS

This application claims priority to U.S. Provisional Application No. 62/853,377, filed on May 28, 2019, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on May 27, 2020, is named 251110000079_seq_listing.txt and is 16,143 bytes in size.

### BACKGROUND

Neonatal hypoxic-ischemic encephalopathy (HIE), a neurological disorder that causes damage to cells in the brain in neonates due to inadequate oxygen supply, is a significant cause of mortality and disability worldwide. The primary causes of neonatal HIE are brain hypoxia and ischemia due to systemic hypoxemia and reduced cerebral blood flow (CBF). Neonatal HIE may cause death in the newborn period or result in what is later recognized as developmental delay, mental retardation, or cerebral palsy (CP). Neonatal HIE is a serious condition that causes significant mortality and morbidity in near-term and term newborns and therefore, it remains a challenge for perinatal medicine.

Neonatal HIE is a significant worldwide public health problem affecting one to two out of 1000 live-births in the developed world, with as much as an eight-fold higher incidence in low and middle income countries with limited access to healthcare [7]. Without treatment, mortality rates in infants with HIE range from 10-60% depending on disease severity, with at least 25% of survivors manifesting significant neurodevelopmental disabilities [8].

Therapeutic hypothermia (HT) is the standard of care treatment for HIE. While HT improves survival and neurodevelopment in newborns with HIE [9], HT only reduces the risk of death or disability by 11 percentage points, i.e., a decrease from 58% to 47% [10]. Childhood outcomes after HT treatment show variable results with benefits waning over time. There was no statistically significant difference between hypothermia and usual care in the rate of a combined end point of death or an IQ score of less than 70 at 6 to 7 years of age [11]. Moreover, among term neonates with moderate or severe HIE, there is no reduction in death or disability (moderate or severe) at 18 months of age with longer or deeper cooling, or both [12]. There may even be an increase in mortality with HT. Further, HT requires specialized equipment and treatment in tertiary care centers, must be initiated within 6 hours of birth, and is associated with undesirable side effects [8, 13].

Current therapeutic strategies for management of neonatal HIE are restricted to therapeutic hypothermia [24]. Over the past several years, attempts to increase the effectiveness and broaden the scope of HT for HIE have not been successful [12]. Inherent variability in disease manifestation makes development of therapeutic interventions for HIE a challenge. For instance, HT may be ineffective or even harmful as a treatment of hypoxia-ischemia sensitized by inflammatory conditions such as chorioamnionitis [21, 25].

No pharmacological adjunct or alternative therapy is currently approved for use in neonatal HIE. Adjunct therapies with non-specific mechanisms of action such as erythropoietin, melatonin, cannabinoids and autologous umbilical cord stem cells are in various phases of study, but none have been approved for use in humans as of yet [26]. Several of these strategies are directed at enhancing repair versus decreasing reperfusion injury.

There is an urgent need for a pharmacological treatment or adjunct for treatment of neonatal HIE.

### BRIEF DESCRIPTION OF THE DRAWINGS

FIG. 1 is a flowchart illustrating an experimental protocol. Term equivalent rat pups at P10-12 were randomly assigned to four different treatment groups: no intervention (control), normothermia (NT), hypothermia (HT), and treatment (PIC1). Experimental animals underwent unilateral carotid ligation followed by exposure to hypoxia (8% O₂/balanced nitrogen, Vannucci model) for 45 minutes. HT animals were placed in open jars in a temperature controlled chamber to maintain a target rectal temperature of 31-32° C. for 6 hours, control, NT, and PIC1 animals were kept in a separate chamber at 37° C. PIC1 animals were injected with two doses of PIC1 (160 mg/kg, i.p.), with the first dose injected at the time of carotid ligation, and the second dose four hours later. After intervention, pups were placed back with the dam, and harvested at different time points.

FIG. 2 is a bar graph showing that PIC1 causes a temporary but significant decrease in systemic complement activity after i.p. injection. In a complement hemolytic assay using sensitized human erythrocytes, plasma from animals injected with PIC1 inhibits systemic complement activation by 70% at 30 minutes after i.p. injection (striped bars), comparable to injection with CVF (hatched bar) with a gradual return to baseline by 8 hours after injection. Treatment with PIC1 resulted in significant systemic complement inhibition at 0.5, 1, 2 and 4 hours after injection when compared to untreated NT animals (white bar). Complement activity at 8, 16 and 48 hours after injection was comparable to untreated controls. In FIG. 2, n=5 animals per group, error bars denote the SEM, and * denotes P<0.05.

FIGS. 3A and 3B show data indicating that treatment with PIC1 decreases brain infarct size after HIE. Brain infarct sizes were compared between groups using TTC, which stains viable portions of the brain red. In FIG. 3A, representative brain slices for NT, HT- and PIC1-treated animals are shown. In FIG. 3B, quantification with Image J showed that PIC1 treatment (striped bar) decreased infarct size by an average of 68% (±4.7%) as compared to NT (white bar; P=0.01). Animals treated with HT (grey bar) also showed a significant decrease in brain infarct size when compared to NT animals. In FIGS. 3A and 3B, n=3 animals per group, error bars denote the SEM, and * denotes P<0.05.

FIGS. 4A-4BB show that TUNEL staining decreased apoptosis after treatment with PIC1. NT animals showed markedly decreased nuclear staining with DAPI (FIGS. 4A and 4D and 4S) when compared to HT- (FIGS. 4G, 4J and 4T) and PIC1- (FIGS. 4M, 4P and 4U) treated animals; nuclear density p=0.01 for PIC1 versus NT (FIG. 4Z); cell count p=0.03 for PIC1 versus NT (FIG. 4AA); cell size p<0.05 for PIC1 versus NT and PIC1 versus HT (FIG. 4BB). NT brains showed significantly increased apoptosis as demonstrated by TUNEL positive cells (FIGS. 4B and 4E), when compared to HT-treated animals (FIGS. 4H and 4K) and PIC1-treated animals (FIGS. 4N and 4Q); p<0.05 for PIC1 versus NT and PIC1 versus HT (FIG. 4Y). NT animals also had significantly more C1q expression (FIGS. 4C and 4F) when compared to HT- (FIGS. 4I and 4L) and PIC1-treated animals (FIGS. 4O and 4R). C1q extensively co-localized with TUNEL in apoptotic cells in NT animals (FIG. 4V), with a qualitative reduction in C1q/TUNEL co-localization in HT (FIG. 4W) and PIC1 (FIG. 4X) treated animals. FIGS. 4A-4BB show ipsilateral hemispheres, cortex and hippocampus, 48 hours after injury. In these figures, the following apply: n=4 per group, 10 fields per brain, magnification is 10× (FIGS. 4A-4C, 4G-4L, and 4M-4O), 20× (FIGS. 4D-4F, 4J-4L, and 4P-4R), 40× (FIGS. 4S-4X); 20× and 40× fields showing cortex; blue=DAPI, green=TUNEL, red=C1q; NT (white bar), HT (gray bar), PIC1 (striped bar). Error bars denote the SEM. The scale bar shown in FIG. 4A is 1.0 mm and also applies to FIGS. 4B, 4C, 4G, 4H, 4I, 4M, 4N and 4O. The scale bar shown in FIG. 4D is 200 μm and also applies to FIGS. 4E, 4F, 4J, 4K, 4L, 4P, 4Q and 4R. The scale bar shown in FIG. 4S is 100 μm and also applies to FIGS. 4T, 4U, 4V, 4W and 4X.

FIGS. 5A-5M show graphs indicating that PIC1 treatment decreases local and systemic complement effector expression. C1q levels in the plasma of HIE animals were significantly higher than those of HT- and PIC1-treated animals at 8, 12 and 24 hours (P<0.05) after the initial insult (FIG. 5A). Levels of systemic C3a (FIG. 5B) and C5a (FIG. 5C) in HIE animals were significantly higher than those of HT- and PIC1-treated animals at 12, 24 and 48 hours (P<0.05) after the initial insult. C1q levels in the brains of NT animals were significantly higher than those of HT- and PIC1-treated animals at 8, 12 and 24 hours (P<0.05) after the initial insult (FIG. 5D). The whole brain expression of C3aR (receptor for C3a) is significantly higher in the PIC1 group when compared to NT animals (P<0.05) (FIGS. 5E, 5G, 5H, 5I and 5J). There were no significant differences in the expression of C5aR (receptor for C5a) between NT and PIC1-treated groups. (FIGS. 5F, 5G, 5K, 5L and 5M). In FIGS. 5A-5M, the following applies: n=7 animals per group, solid lines/white bars—NT; dotted lines/grey bars—HT; dashed line/striped bar—PIC1. FIGS. 5H-5M depict the cortex at 20× magnification, error bars denote ±SEM, and * denotes P<0.05.

FIGS. 6A-6GG illustrate that PIC1 treatment decreases neuroinflammation after HIE. PIC1-treated animals (FIGS. 6C, 6F and 6U) demonstrate a higher neuronal density when compared to HIE (FIGS. 6A, 6D and 6S) and HT-treated (FIGS. 6B, 6E and 6T) animals (FIG. 6BB: P<0.05 HIE versus PIC1 and HT versus PIC1). PIC1-treated animals show reduced microglial numbers and size (FIGS. 6I, 6L and 6X), and reduced astrogliosis (FIGS. 60, 6R and 6AA), when compared to HIE animals (FIGS. 6G, 6J, 6M, 6P, 6V, 6Y, 6CC and 6DD: P<0.05 HIE versus PIC1). C1q primarily co-localizes in the microglia (FIGS. 6G, 6H and 6I), with scant co-localization in neurons (FIGS. 6A, 6B and 6C) and astrocytes (FIGS. 6M, 6N and 6O). C3 primarily co-localizes in the microglia (FIGS. 6J, 6K and 6L) and astrocytes (FIGS. 6P, 6Q and 6R), with scant co-localization in neurons (FIGS. 6D, 6E and 6F). C9 extensively co-localizes with neurons (FIGS. 6S, 6T and 6U), microglia (FIGS. 6V, 6W and 6X) and astrocytes (FIGS. 6Y, 6Z and 6AA). Quantification shows significant reduction in C1q expression in PIC1 animals when compared to HIE and HT animals. (FIG. 6EE. P<0.05 HIE versus PIC1 and HT versus PIC1). PIC1-treated animals showed decreased expression in C3 and C9 when compared to HIE animals. (FIGS. 6FF and 6GG: P<0.05 HIE versus PIC1). FIGS. 6A-6GG show ipsilateral hemispheres, cortex and hippocampus, 48 hours after injury. In these figures, the following applies: n=4 per group, 10 fields per brain, magnification is 20×; green=NeuN (FIGS. 6A-6C), C3 (FIGS. 6D-6F), Iba1 (FIGS. 6G-6L), GFAP (FIGS. 6M-6R), C9 (FIGS. 6S-6AA); red=C1q (FIGS. 6A-6C and 6G-60), NeuN (FIGS. 6D-6F and 6S-6U), C3 (FIGS. 6J-6L and 6P-6R), Iba1 (FIGS. 6V-6X), GFAP (FIGS. 6Y-6AA); the white bar represents HIE, the gray bar represents HT, the striped bar represents PIC1.

FIGS. 7A-7D show that PIC1 treatment improves motor, spatial memory and cognitive outcomes after HIE. Spatial learning and memory was evaluated in a Barnes maze. PIC1-treated animals demonstrated a significantly shorter latency (FIG. 7A), and committed fewer errors (FIG. 7B) in finding the escape hole when compared to HIE and HT animals. Fine motor coordination and balance was assessed by the beam walk assay. PIC1-treated animals performed significantly better than HIE animals on the 0.75 inch (FIG. 7C) and 0.25 inch (FIG. 7D) beams. In FIGS. 7A-7D, the following apply: N=7 animals per group; Red: HIE, Blue: HT, Green: PIC1. Notched box-plots: Box—interquartile range (25-75th percentile); Horizontal line—median; Notch—confidence. If the notches in two boxes do not overlap, there is 95% confidence their medians differ.

FIG. 8 is a graph showing the results of the novel object recognition test for cognitive ability. PIC1-treated animals spent significantly higher proportion of time exploring the novel object when compared to NT and HT animals. In FIG. 8, the following applies: N=7 animals per group, error bars denote the SEM, and * denotes p<0.05.

FIGS. 9A-9C illustrate that the contralateral hemisphere in NT animals does not show evidence of neuronal injury or gliosis. The contralateral cortex of NT brains was stained for microglia (FIG. 9A), astrocytes (FIG. 9B) and neurons (FIG. 9C). There is scant staining for microglia and astrocytes, and robust staining for neurons in the contralateral hemisphere, confirming that there is no neuronal damage or gliosis. Microscopy was performed at 20× magnification.

FIGS. 10A-10H illustrate that PIC1 treatment decreases microglial activation in HIE. TMEM119+ is a microglial specific marker. Compared to NT (FIG. 10A) and HT (FIG. 10B), PIC1 treatment (FIG. 10C) results in almost a three-fold decrease in TMEM119 expression in the cortex (FIG. 10D). Compared to NT (FIG. 10E) and HT (FIG. 10F) animals, the cortex of PIC1 (FIG. 10G) animals showed an almost two-fold decrease in combined Iba1/TMEM119 staining (FIG. 10H). FIGS. 10A-10H show ipsilateral hemispheres, cortex, 48 hours after injury; n=4 per group, 10 fields per brain, magnification is 20×, green represents Iba1; and red represents TMEM119.

FIGS. 11A-11D shows that HT and PIC1 treatment each decreases oxidant stress in HIE. When compared to NT (FIG. 11A) animals, MPO (Myeloperoxidase) expression in HT (FIG. 11B) and PIC1-treated (FIG. 11C) brains is significantly decreased (FIG. 11D) (P<0.01). HT animals show the greatest decrease in MPO expression. MPO extensively co-localizes with microglia. FIGS. 11A-11D show ipsilateral hemispheres, cortex, 48 hours after injury; n=4 per group, 10 fields per brain, magnification is 40×; green represents Iba1; and red represents MPO.

FIG. 12 shows the body weight of pooled gender Wistar rat pups, at the age of 10±1 days. Data are presented as mean+SEM. Group 1: H/I+normo, n=10; Group 2: H/I+hypo, n=10; Group 3: H/I+PIC1+normo, n=10; Group 4: H/I+PIC1+hypo, n=10; Group 5: Naive+normo, n=5. No significant differences were observed between the groups within the study.

FIG. 13 shows the body weight of female gender Wistar rat pups, at the age of 10±1 days. Data are presented as mean+SEM. Group 1: H/I+normo, n=4; Group 2: H/I+hypo, n=3; Group 3: H/I+PIC1+normo, n=6; Group 4: H/I+PIC1+hypo, n=3; Group 5: Naive+normo, n=1. No significant differences were observed between the groups within the study.

FIG. 14 shows the body weight of male gender Wistar rat pups, at the age of 10±1 days. Data are presented as mean+SEM. Group 1: H/I+normo, n=10; Group 2: H/I+hypo, n=10; Group 3: H/I+PIC1+normo, n=10; Group 4: H/I+PIC1+hypo, n=10; Group 5: Naive+normo, n=5. No significant differences were observed between the groups within the study.

FIG. 15 shows the effects of normothermia (36+/−1° C. rat internal temperature) and PIC1 treatment on lesion T2 values of neonatal Wistar rat pups (pooled gender) subjected to brain hypoxia/ischemia (H/I). Data are presented as mean±SEM. Group 1: H/I+normo, n=9; Group 2: H/I+hypo, n=10; Group 3: H/I+PIC1+normo, n=9; Group 4: H/I+PIC1+hypo WT, n=9. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 16 shows the effects of normothermia (36+/−1° C. rat internal temperature) and PIC1 treatment on lesion T2 values of neonatal Wistar rat pups (females) subjected to brain hypoxia/ischemia (WI). Data are presented as mean±SEM. Group 1: H/I+normo, n=3; Group 2: H/I+hypo, n=3; Group 3: H/I+PIC1+normo, n=5; Group 4: H/I+PIC1+hypo WT, n=3. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 17 shows the effects of normothermia (36+/−1° C. rat internal temperature) and PIC1 treatment on lesion T2 values of neonatal Wistar rat pups (males) subjected to brain hypoxia/ischemia (H/I). Data are presented as mean±SEM. Group 1: H/I+normo, n=6; Group 2: H/I+hypo, n=7; Group 3: H/I+PIC1+normo, n=4; Group 4: H/I+PIC1+hypo WT, n=6. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 18 shows the effects of normothermia (36+/−1° C. rat internal temperature) and PIC1 treatment on control T2 values of neonatal Wistar rat pups (pooled gender) subjected to brain hypoxia/ischemia (WI). Data are presented as mean±SEM (Group 1: H/I+normo, n=9; Group 2: H/I+hypo, n=10; Group 3: H/I+PIC1+normo, n=9; Group 4: H/I+PIC1+hypo WT, n=9; Group 5: Naive+normo, n=5. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 19 shows the effects of normothermia (36+/−1° C. rat internal temperature) and PIC1 treatment on control T2 values of neonatal Wistar rat pups (females) subjected to brain hypoxia/ischemia (WI). Data are presented as mean±SEM (Group 1: H/I+normo, n=3; Group 2: H/I+hypo, n=3; Group 3: H/I+PIC1+normo, n=5; Group 4: H/I+PIC1+hypo WT, n=3; Group 5: Naive+normo, n=1. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 20 shows the effects of normothermia (36+/−1° C. rat internal temperature) and PIC1 treatment on control T2 values of neonatal Wistar rat pups (males) subjected to brain hypoxia/ischemia (WI). Data are presented as mean±SEM (Group 1: H/I+normo, n=6; Group 2: H/I+hypo, n=7; Group 3: H/I+PIC1+normo, n=4; Group 4: H/I+PIC1+hypo WT, n=6; Group 5: Naive+normo, n=4. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 21 shows the effects of normothermia (36+/−1° C. rat internal temperature) and PIC1 treatment on lesion volume of neonatal Wistar rat pups (pooled gender) subjected to brain hypoxia/ischemia (WI). Data are presented as mean±SEM. Group 1: H/I+normo, n=9; Group 2: H/I+hypo, n=10; Group 3: H/I+PIC1+normo, n=9; Group 4: H/I+PIC1+hypo WT, n=9. There was a significant difference at 24 h when Group 2 was compared to Group 4. (#p<0.05; RM two-way ANOVA, Sidak's post hoc).

FIG. 22 shows the effects of normothermia (36+/−1° C. rat internal temperature) and PIC1 treatment on lesion volume of neonatal Wistar rat pups (females) subjected to brain hypoxia/ischemia (H/I). Data are presented as mean±SEM (Group 1: H/I+normo, n=3; Group 2: H/I+hypo, n=3; Group 3: H/I+PIC1+normo, n=5; Group 4: H/I+PIC1+hypo WT, n=3. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 23 shows the effects of normothermia (36+/−1° C. rat internal temperature) and PIC1 treatment on lesion volume of neonatal Wistar rat pups (males) subjected to brain hypoxia/ischemia (H/I). Data are presented as mean±SEM. Group 1: H/I+normo, n=6; Group 2: H/I+hypo, n=7; Group 3: H/I+PIC1+normo, n=4; Group 4: H/I+PIC1+hypo WT, n=6. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 24 shows the effects of normothermia (36+/−1° C. rat internal temperature) and PIC1 treatment on edema percentage of neonatal Wistar rat pups (pooled gender) subjected to brain hypoxia/ischemia (H/I). Data are presented as mean±SEM. (Group 1: H/I+normo, n=9; Group 2: H/I+hypo, n=10; Group 3: H/I+PIC1+normo, n=9; Group 4: H/I+PIC1+hypo WT, n=9; Group 5: Naive+normo, n=5. There was a significant difference at Day 21 when Group 3 was compared to Group 2. (##p<0.01; RM two-way ANOVA, Sidak's post hoc).

FIG. 25 shows the effects of normothermia (36+/−1° C. rat internal temperature) and PIC1 treatment on edema percentage of neonatal Wistar rat pups (female) subjected to brain hypoxia/ischemia (WI). Group 1: H/I+normo, n=3; Group 2: H/I+hypo, n=3; Group 3: H/I+PIC1+normo, n=5; Group 4: H/I+PIC1+hypo WT, n=9, 3, 6; Group 5: Naive+normo, n=1. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 26 shows the effects of normothermia (36+/−1° C. rat internal temperature) and PIC1 treatment on edema percentage of neonatal Wistar rat pups (male) subjected to brain hypoxia/ischemia (WI). Group 1: H/I+normo, n=6; Group 2: H/I+hypo, n=7; Group 3: H/I+PIC1+normo, n=4; Group 4: H/I+PIC1+hypo WT, n=6; Group 5: Naive+normo, n=4. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 27 shows the effects of normothermia (36+/−1° C. rat internal temperature) and PIC1 treatment on balance beam performance of neonatal Wistar rat pups (pooled gender) subjected to brain hypoxia/ischemia (WI). Data are presented as mean±SEM. Group 1: H/I+normo, n=10; Group 2: H/I+hypo, n=10; Group 3: H/I+PIC1+normo, n=10; Group 4: H/I+PIC1+hypo WT, n=10; Group 5: Naive+normo, n=5. There was a significant difference observed between Group 3 and Group 4 versus Group 1 (* p<0.05, Welch's t-test).

FIG. 28 shows the effects of normothermia (36+/−1° C. rat internal temperature) and PIC1 treatment on balance beam performance of neonatal Wistar rat pups (females) subjected to brain hypoxia/ischemia (WI). Data are presented as mean±SEM. Group 1: H/I+normo, n=4; Group 2: H/I+hypo, n=3; Group 3: H/I+PIC1+normo, n=6; Group 4: H/I+PIC1+hypo WT, n=3; Group 5: Naive+normo, n=1. No statistical significances between any of the groups comparison; Group 1 versus Group 3; Group 1 versus Group 4; Group 2 versus Group 3; Group 2 versus Group 4. (p>0.05, Welch's t-test).

FIG. 29 shows the effects of normothermia (36+/−1° C. rat internal temperature) and PIC1 treatment on balance beam performance of neonatal Wistar rat pups (males) subjected to brain hypoxia/ischemia (H/I). Data are presented as mean±SEM. Group 1: H/I+normo, n=6; Group 2: H/I+hypo, n=7; Group 3: H/I+PIC1+normo, n=4; Group 4: H/I+PIC1+hypo WT, n=7; Group 5: Naive+normo, n=4. There was a significant difference observed between Group 3 and Group 4 versus Group 1 (* p<0.05, Welch's t-test).

FIG. 30 shows the effects of normothermia (36+/−1° C. rat internal temperature) and PIC1 treatment on balance beam performance of neonatal Wistar rat pups (pooled gender) subjected to brain hypoxia/ischemia (H/I). Data are presented as mean±SEM (Group 1: H/I+normo, n=10; Group 2: H/I+hypo, n=10; Group 3: H/I+PIC1+normo, n=10; Group 4: H/I+PIC1+hypo WT, n=10; Group 5: Naive+normo, n=5. No statistical significances between any of the groups comparison; Group 1 versus Group 3; Group 1 versus Group 4; Group 2 versus Group 3; Group 2 versus Group 4 (p>0.05, Welch's t-test).

FIG. 31 shows the effects of normothermia (36+/−1° C. rat internal temperature) and PIC1 treatment on balance beam performance of neonatal Wistar rat pups subjected to brain hypoxia/ischemia (H/I). Data are presented as mean±SEM. Group 1: H/I+normo, n=4; Group 2: H/I+hypo, n=3; Group 3: H/I+PIC1+normo, n=5; Group 4: H/I+PIC1+hypo WT, n=2; Group 5: Naive+normo, n=1. No statistical significances between any of the groups comparison; Group 1 versus Group 3; Group 1 versus Group 4; Group 2 versus Group 3; Group 2 versus Group 4 (p>0.05, Welch's t-test).

FIG. 32 shows the effects of normothermia (36+/−1° C. rat internal temperature) and PIC1 treatment on balance beam performance of neonatal Wistar rat pups subjected to brain hypoxia/ischemia (H/I). Data are presented as mean±SEM. Group 1: H/I+normo, n=6; Group 2: H/I+hypo, n=7; Group 3: H/I+PIC1+normo, n=5; Group 4: H/I+PIC1+hypo WT, n=8; Group 5: Naive+normo, n=4. No statistical significances between any of the groups comparison; Group 1 versus Group 3; Group 1 versus Group 4; Group 2 versus Group 3; Group 2 versus Group 4 (p>0.05, Welch's t-test).

FIG. 33 shows the effects of normothermia (36+/−1° C. rat internal temperature) and PIC1 treatment on T-maze performance (time spent in separate areas of the maze) of neonatal Wistar rat pups (pooled gender) subjected to brain hypoxia/ischemia (WI). Data are presented as mean+SEM. Group 1: H/I+normo, n=10; Group 2: H/I+hypo, n=10; Group 3: H/I+PIC1+normo, n=10; Group 4: H/I+PIC1+hypo, n=10; Group 5: Naive+normo, n=5. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 34 shows the effects of normothermia (36+/−1° C. rat internal temperature) and PIC1 treatment on T-maze performance (time spent in separate areas of the maze) of neonatal Wistar rat pups (females) subjected to brain hypoxia/ischemia (WI). Data are presented as mean+SEM. Group 1: H/I+normo, n=4; Group 2: H/I+hypo, n=3; Group 3: H/I+PIC1+normo, n=6; Group 4: H/I+PIC1+hypo, n=3; Group 5: Naive+normo, n=1. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

FIG. 35 shows the effects of normothermia (36+/−1° C. rat internal temperature) and PIC1 treatment on T-maze performance (time spent in separate areas of the maze) of neonatal Wistar rat pups (males) subjected to brain hypoxia/ischemia (H/I). Data are presented as mean+SEM. Group 1: H/I+normo, n=6; Group 2: H/I+hypo, n=7; Group 3: H/I+PIC1+normo, n=4; Group 4: H/I+PIC1+hypo, n=7; Group 5: Naive+normo, n=4. No statistical significances between any of the groups comparison; Group 1 versus Group 3; Group 1 versus Group 4; Group 2 versus Group 3; Group 2 versus Group 4 (p>0.05, RM two-way ANOVA, Sidak's post hoc).

### SUMMARY OF THE INVENTION

In one aspect is provided a method of treating hypoxic ischemic encephalopathy (HIE) in a subject, the method comprising administering a therapeutically effective amount of a classical complement pathway inhibitor to the subject, where the subject is a neonate. In some embodiments, the classical complement pathway inhibitor is administered once to the subject within 24 hours of onset of the HIE. In some embodiments, the classical complement pathway inhibitor is administered to the subject once a day, twice a day, three times a day, four times a day, five times a day, or six times a day for 1, 2, 3, 4, 5, 6, or 7 days after onset of the HIE.

In various embodiments, the classical complement pathway inhibitor inhibits inflammatory immune cell (e.g., microglia) recruitment, inflammatory immune cell (e.g., microglia) activation, and/or MPO. In various embodiments, the classical complement pathway inhibitor inhibits inflammation. In various embodiments, the classical complement pathway inhibitor is an antioxidant.

In another aspect is provided a method for improving cognition and memory of a subject suffering from a neurodegenerative disease caused by neonatal hypoxic ischemic encephalopathy (HIE), the method comprising administering a therapeutically effective amount of a classical complement pathway inhibitor to the subject.

In some embodiments of the above aspects, the method is effective to inhibit C1q-mediated activation of the classical complement pathway in the brain of the subject. In some embodiments, the method is effective to reduce the level of one or more of C1q, C3, C5a, or C9 in the brain of the subject. In some embodiments, the method is effective to increase the expression of C3a receptor (C3aR) in the brain of the subject. In some embodiments, the method is effective to reduce systemic inflammation. In some embodiments, the method is effective to reduce oxidant stress in the brain of the subject.

In some embodiments of the above aspects, the method is effective to improve one or more of fine motor performance, spatial memory and cognition in the subject, as compared to another subject not treated with PIC1. In some embodiments, the method is effective to treat or prevent cerebral palsy.

In some embodiments of the above aspects, the classical complement pathway inhibitor is administered parenterally.

In various embodiments, the classical complement pathway inhibitor is administered in combination with therapeutic hypothermia.

In some embodiments of the above aspects, the classical complement pathway inhibitor is PIC1. In some embodiments, the PIC1 peptide comprises one or more PEG moieties. In some embodiments, the PIC1 is a peptide comprising at least about 90% sequence identity to at least one amino acid sequence selected from the group consisting of SEQ ID NO: 3-47. In some embodiments, the PIC1 is PA-dPEG24. In some embodiments, the PA-dPEG24 comprises the sequence of IALILEPICCQERAA-dPEG24 (SEQ ID NO: 19).

### DETAILED DESCRIPTION OF THE INVENTION

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention. When certain terms are explained or defined in connection with a particular aspect or embodiment, such connotation is meant to apply throughout this specification, i.e., also for other aspects or embodiments, unless otherwise specified or unless the context clearly dictates otherwise.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the terms “treat” or “treatment” refer to both therapeutic treatment and prophylactic or preventative measures, wherein the object is to prevent or slow down (lessen) an undesired physiological change or disorder, such as the development of a neurological disorder. Beneficial or desired clinical results include, but are not limited to, prevention of a disorder, reducing the incidence of a disorder, alleviation of symptoms associated with a disorder, diminishment of extent of a disorder, stabilizing (i.e., not worsening) the state of a disorder, delaying or slowing progression of a disorder, amelioration or palliation of the state of a disorder, remission (whether partial or total), whether detectable or undetectable, or combinations thereof. “Treatment” can also mean prolonging survival as compared to expected survival if not receiving treatment.

Except when noted, “subject” or “patient” are used interchangeably and refer to animals, preferably warm-blooded animals, more preferably vertebrates, even more preferably mammals, still more preferably primates, and specifically include human patients and non-human mammals and primates. “Mammalian” subjects refer to any animal classified as such and include, but are not limited to, humans, domestic animals, commercial animals, farm animals, zoo animals, sport animals, pet and experimental animals such as dogs, cats, guinea pigs, rabbits, rats, mice, horses, cattle, cows, wolves, lions, tigers, horses, donkeys, zebras, cows, pigs, sheep, deer, giraffes, and rodents such as mice, rats, hamsters and guinea pigs. Preferred subjects are human patients.

The term “therapeutically effective amount” as used herein refers to an amount of a PIC1 compound or a pharmaceutical composition as taught herein effective to treat a neurological disorder in a subject, i.e., to obtain a desired local or systemic effect and performance. The term thus refers to the quantity of PIC1 compound or pharmaceutical composition that elicits the biological or medicinal response in a tissue, system, animal, or human that is being sought by a researcher, veterinarian, medical doctor or other clinician. In particular, the term refers to the quantity of PIC1 compound or pharmaceutical composition as taught herein which is necessary to prevent, cure, ameliorate, or at least minimize the clinical impairment, symptoms, or complications associated with a neurological disorder in either a single or multiple doses.

The terms “neurodegenerative disease” and “neurodegenerative disorder” refer to a neurological disorder characterized by the progressive loss of structure or function of neurons, including death of neurons.

In one aspect is provided a method of treating hypoxic ischemic encephalopathy (HIE) in a subject, the method comprising administering a therapeutically effective amount of a classical complement pathway inhibitor to the subject, where the subject is a neonate. The classical complement pathway inhibitor may be PIC1 or a PIC1 variant. As described throughout the application, administering PIC1 or a PIC1 variant to a neonate with HIE can be effective to prevent neuron loss, synapse elimination, and brain damage. These in turn can prevent or ameliorate any development of cerebral palsy and other conditions that may arise from HIE.

In another aspect is provided a method for improving cognition and memory of a subject suffering from a neurodegenerative disease or disorder caused by neonatal hypoxic ischemic encephalopathy (HIE), the method comprising administering a therapeutically effective amount of a classical complement pathway inhibitor to the subject. The neurodegenerative disease or disorder may be cerebral palsy, for example.

In various embodiments, HIE may result from oxygen deprivation due to a reduction in the supply of oxygen to the brain. HIE can also be caused by, or aggravated by, an ischemia-reperfusion injury that occurs subsequent to the hypoxia or anoxia.

Examples of PIC1 and PIC1 variants include, but are not limited to, the peptides listed in Table 1.

TABLE 1

| Peptide name | Peptide sequence |
|---|---|
| PA | IALILEPICCQERAA |
| | (SEQ ID NO: 1) |
| PA-I1Sar | (Sar)ALILEPICCQERAA |
| | (SEQ ID NO: 2) |
| PA-A2Sar | I(Sar)LILEPICCQERAA |
| | (SEQ ID NO: 3) |
| PA-L3 Sar | IA(Sar)ILEPICCQERAA |
| | (SEQ ID NO: 4) |
| PA-I4Sar | IAL(Sar)LEPICCQERAA |
| | (SEQ ID NO: 5) |
| PA-L5Sar | IALI(Sar)EPICCQERAA |
| | (SEQ ID NO: 6) |
| PA-E6Sar | IALIL(Sar)PICCQERAA |
| | (SEQ ID NO: 7) |
| PA-P7Sar | IALILE(Sar)ICCQERAA |
| | (SEQ ID NO: 8) |
| PA-I8Sar | IALILEP(Sar)CCQERAA |
| | (SEQ ID NO: 9) |
| PA-C9Sar | IALILEPI(Sar)CQERAA |
| | (SEQ ID NO: 10) |
| PA-C10Sar | IALILEPIC(Sar)QERAA |
| | (SEQ ID NO: 11) |
| PA-Q11Sar | ALILEPICC(Sar)ERAA |
| | (SEQ ID NO: 12) |
| PA-E12Sar | IALILEPICCQ(Sar)RAA |
| | (SEQ ID NO: 13) |
| PA-R13Sar | IALILEPICCQE(Sar)AA |
| | (SEQ ID NO: 14) |
| PA-A14Sar | IALILEPICCQER(Sar)A |
| | (SEQ ID NO: 15) |
| PA-A15Sar | IALILEPICCQERA(Sar) |
| | (SEQ ID NO: 16) |
| dPEG24-PA-dPEG24 | dPEG24-IALILEPICCQERAA-dPEG24 |
| | (SEQ ID NO: 17) |
| dPEG24-PA | dPEG24-IALILEPICCQERAA |
| | (SEQ ID NO: 18) |
| PA-dPEG24 | IALILEPICCQERAA-dPEG24 |
| | (SEQ ID NO: 19) |
| PA-dPEG20 | IALILEPICCQERAA-dPEG20 |
| | (SEQ ID NO: 20) |
| PA-dPEG16 | IALILEPICCQERAA-dPEG16 |
| | (SEQ ID NO: 21) |
| PA-dPEG12 | IALILEPICCQERAA-dPEG12 |
| | (SEQ ID NO: 22) |
| PA-dPEG08 | IALILEPICCQERAA-dPEG08 |
| | (SEQ ID NO: 23) |
| PA-dPEG06 | IALILEPICCQERAA-dPEG06 |
| | (SEQ ID NO: 24) |
| PA-dPEG04 | IALILEPICCQERAA-dPEG04 |
| | (SEQ ID NO: 25) |
| PA-dPEG03 | IALILEPICCQERAA-dPEG03 |
| | (SEQ ID NO: 26) |
| PA-dPEG02 | IALILEPICCQERAA-dPEG02 |
| | (SEQ ID NO: 27) |
| PA-C9SarC10A | IALILEPI(Sar)AQERAA |
| | (SEQ ID NO: 28) |
| PA-C9SarΔ10 | IALILEPI(Sar)QERAA |
| | (SEQ ID NO: 29) |
| PA-P7SarC9Sar | IALILE(Sar)I(Sar)CQERAA |
| | (SEQ ID NO: 30) |
| PA-E6Sar-dPEG24 | IALIL(Sar)PICCQERAA-dPEG24 |
| | (SEQ ID NO: 31) |
| PA-Q11Sar-dPEG24 | IALILEPICC(Sar)ERAA-dPEG24 |
| | (SEQ ID NO: 32) |
| PA-R13Sar-dPEG24 | IALILEPICCQE(Sar)AA-dPEG24 |
| | (SEQ ID NO: 33) |
| PA-A14Sar-dPEG24 | IALILEPICCQER(Sar)A-dPEG24 |
| | (SEQ ID NO: 34) |
| E6SarP7Sar | IALIL(Sar)(Sar)ICCQERAA |
| | (SEQ ID NO: 35) |
| E6SarC9Sar | IALIL(Sar)PI(Sar)CQERAA |
| | (SEQ ID NO: 36) |
| Q11SarP7Sar | IALILE(Sar)ICC(Sar)ERAA |
| | (SEQ ID NO: 37) |
| Q11SarC9Sar | IALILEPI(Sar)C(Sar)ERAA |
| | (SEQ ID NO: 38) |
| R13SarP7Sar | IALILE(Sar)ICCQE(Sar)AA |
| | (SEQ ID NO: 39) |
| R13SarC9Sar | IALILEPI(Sar)CQE(Sar)AA |
| | (SEQ ID NO: 40) |
| Al4SarP7Sar | IALILE(Sar)ICCQER(Sar)A |
| | (SEQ ID NO: 41) |
| Al4SarC9Sar | IALILEPI(Sar)CQER(Sar)A |
| | (SEQ ID NO: 42) |
| E6AE12A-dPEG24 | IALILAPICCQARAA-dPEG24 |
| | (SEQ ID NO: 43) |
| E6AE12AC9Sar | IALILAPI(Sar)CQARAA |
| | (SEQ ID NO: 44) |
| E6AE12AP7Sar | IALILA(Sar)ICCQARAA |
| | (SEQ ID NO: 45) |

In some embodiments, PIC1 comprises one or more PEG moieties. The PEG moieties may be attached to the N-terminus, the C-terminus, or both the N-terminus and C-terminus by PEGylation. In one or more embodiments, 24 PEG moieties are attached to the N-terminus. In one or more embodiments, 24 PEG moieties are attached to the C-terminus. In one or more embodiments, 24 PEG moieties are attached to the N-terminus and to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the N-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to both the N-terminus and the C-terminus.

The PIC1 peptide may be a synthetic peptide. A synthetic peptide is prepared in vitro. Synthetic peptides can be prepared according to various methods known in the art. For example, a synthetic peptide can be prepared by sequentially coupling individual amino acids to form the peptide. In some embodiments, the carboxyl group of individual amino acids is sequentially coupled to the amino terminus of a growing peptide chain. Protecting groups can be used to prevent unwanted side reactions from occurring during the coupling process. Peptide synthesis can occur in liquid phase or in solid phase.

Exemplary PIC1 peptides include, but are not limited to, PA-dPEG24 (a peptide comprising the polar assortant (PA) sequence and 24 PEG moieties at the C-terminus), PA-dPEG20 (comprising 20 PEG moieties at the C-terminus), PA-dPEG16 (comprising 16 PEG moieties at the C-terminus), PA-dPEG12 (comprising 12 PEG moieties at the C-terminus), PA-dPEG08 (comprising 8 PEG moieties at the C-terminus), PA-dPEG06 (comprising 6 PEG moieties at the C-terminus), PA-dPEG04 (comprising 4 PEG moieties at the C-terminus), PA-dPEG03 (comprising 3 PEG moieties at the C-terminus), and PA-dPEG02 (comprising two PEG moieties at the C-terminus).

In HIE, the entire brain is deprived of an adequate oxygen supply, but the deprivation is not total. The inadequate oxygen supply may be due to hypoxemia, i.e., reduced oxygen content in the blood. The inadequate oxygen supply may involve ischemia. Some damage to brain tissue may occur from initial hypoxia. Additional and generally greater brain tissue injury may occur during the reperfusion phase of ischemia-reperfusion injury. Damage that arises from ischemia-reperfusion injury can be treated with the various compositions and methods described herein. Without wishing to be bound by theory, injury during the reperfusion phase involves inflammatory and oxidant stress mechanisms. The various compositions and methods described herein may be effective to moderate disease severity, such as by reducing inflammation and oxidative stress.

In certain embodiments, HIE affects at least the hippocampus, at least the cerebral cortex, or at least both the hippocampus and the cerebral cortex.

HIE may be caused by a hypoxic injury. The therapeutically effective amount of PIC1 may be effective to treat a hypoxic injury in a neonate, or in a subject who sustained a hypoxic injury as a neonate. HIE may be caused by an anoxic injury. The therapeutically effective amount of PIC1 may be effective to treat an anoxic injury, e.g., in a neonate or in a subject who sustained the anoxic injury as a neonate.

The therapeutically effective amount of the peptide compound varies depending on several factors, such as the severity of the condition, the time of administration, the route of administration, the rate of excretion of the compound employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The hypoxic injury or the anoxic injury may affect one or more localized areas of the brain. The hypoxic injury or the anoxic injury may affect the entire brain. The hypoxic brain injury may affect at least the hippocampus. The hypoxic brain injury may affect at least the cerebral cortex. The hypoxic brain injury may affect at least the hippocampus and the cerebral cortex. The anoxic brain injury may affect at least the hippocampus. The anoxic brain injury may affect at least the cerebral cortex. The anoxic brain injury may affect at least the hippocampus and the cerebral cortex.

In various embodiments, the subject is an infant and HIE is neonatal HIE. Treatment of a subject with PIC1 may be effective to protect against brain damage. Treatment of a subject with PIC1 may be effective to prevent one or more of the following: developmental delay, mental retardation, and cerebral palsy (CP). The prevention of CP may provide for improvement in one or more of motor, behavior, and cognitive function. CP may be characterized by chronic disorders of movement or posture, and may be accompanied by seizure disorders, sensory impairment and/or cognitive limitation.

Without wishing to be bound by theory, activation of the classical pathway of complement during the reperfusion phase may be instrumental in the pathogenesis of HIE. The complement system is the most potent inflammatory cascade in humans and plays a major role in innate immune defense as well as many inflammatory diseases including ischemia-reperfusion injuries (IRI) such as hypoxic ischemic encephalopathy (HIE) [1]. Inflammation mediated by the classical complement cascade may play a major role in the pathogenesis of HIE.

In some embodiments of the above aspects, the method is effective to inhibit C1q-mediated activation of the classical complement pathway in the brain of the subject. C1q protein can be found in a C1 complex with two C1r proteins and two C1s proteins. C1q binding to apoptotic cells may activate the classical complement pathway. Experimental studies have shown that C1q is highly expressed in the brain following ischemia [3], and that classical complement pathway activation via C1q generates pro-inflammatory mediators such as C5a, which are associated with HI brain injury [4]. Additionally, deletion of C1q not only reduces brain infarction and neurofunctional deficit but also results in protection of mitochondrial respiration, indicating a role for classical complement activation and brain oxidative stress [5, 6]. Ischemic brains may have significantly greater deposition of C1q, C3 and C3-split products, and C9, each of which can be associated with cerebral damage [5, 14].

The peptide inhibitor of complement C1 (PIC1) may inhibit the classical pathway of complement. Experimental data provided herein shows that PIC1 is neuroprotective in a rat model of neonatal HIE. Treatment of a subject undergoing HIE with PIC1 can improve histopathological and neurocognitive outcomes.

In some embodiments, the method is effective to reduce the level of one or more of C1q, C3, C5a, or C9 in the brain of the subject. Treatment with PIC1 can reduce systemic inflammation as measured by C1q and C5a levels. Microglia are morphologically, immunophenotypically and functionally related to cells of the monocyte/macrophage lineage. Acute CNS insult is associated with activation of microglia.

In some embodiments, the method is effective to increase the expression of C3a receptor (C3aR) in the brain of the subject. C3aR is a G protein-coupled receptor.

Treatment with PIC1 can also reduce oxidant stress, e.g., as measured by MPO expression. Treatment with PIC1 can also reduce inflammation as measured by local C1q levels and microglia. PIC1-treated animals demonstrate significantly decreased neuronal loss and apoptosis. Lastly, PIC1 treatment improved fine motor performance, spatial memory and cognition in the setting of HIE insult. Described herein is the first study showing the therapeutic potential of a classical pathway complement inhibitor in HIE.

In various embodiments, the therapeutically effective amount of PIC1 is effective to treat ischemia-reperfusion injury that occurs subsequent to the hypoxia or anoxia. HIE may involve a ischemia-reperfusion injury, where tissue hypoxia and microvascular dysfunction are further complicated by subsequent reperfusion, activating innate and adaptive immune and inflammatory responses, which worsen tissue injury and destruction [20, 21]. Administration of PIC1 to an infant suffering from HIE may be effective to decrease C1q expression. By reducing C1q expression, PIC1 may be effective to reduce activation of microglia and neurodegeneration that can occur during ischemia-reperfusion injury. Experimental data provided herein shows that C1q is primarily expressed on microglia after HIE, and that animals treated with PIC1 have reduced numbers of microglia and decreased C1q expression.

In various embodiments, PIC1 is administered parenterally, e.g., such as by intravenous, intracerebral, intracerebroventricular, intramuscular, or subcutaneous injection, or by intravenous infusion.

In various embodiments, PIC1 may be administered once to the subject. In various embodiments, PIC1 may be administered to the subject once a day, twice a day, three times a day, four times a day, five times a day, or six times a day for 1, 2, 3, 4, 5, 6, or 7 days after HIE onset. In various embodiments, PIC1 is administered during HIE, once within 6 hours of HIE onset, once within 12 hours of HIE onset, once within 18 hours of HIE onset, and once within 24 hours of HIE onset. In various embodiments, PIC1 is administered as a maintenance dose once per week, or once every two weeks, after PIC1 onset. Without wishing to be bound by theory, it may be beneficial to administer PIC1 to the subject only for a short period of time during and after an episode of HIE. Complement inhibition in the growing brain may provide for multiple beneficial roles in brain development, including inhibiting neuroinflammation and promoting healing. C1q and C3 are required for retinogeniculate and cortical synaptic pruning during murine CNS development [27, 28]. However, these developmental processes are precisely controlled by various regulators [29]. During an infection and other types of injury, the amplification of the complement cascade produces pro-inflammatory effector molecules such as C5a, altering the balance between activation and regulation, which can result in host tissue damage [30].

However, prolonged complement inhibition may interfere with the complement-mediated repair of damaged host tissue. In a mouse model of spinal cord injury, complement inhibition with a C5a receptor antagonist was protective in the acute phase, but deleterious in the post-acute phase of injury [31].

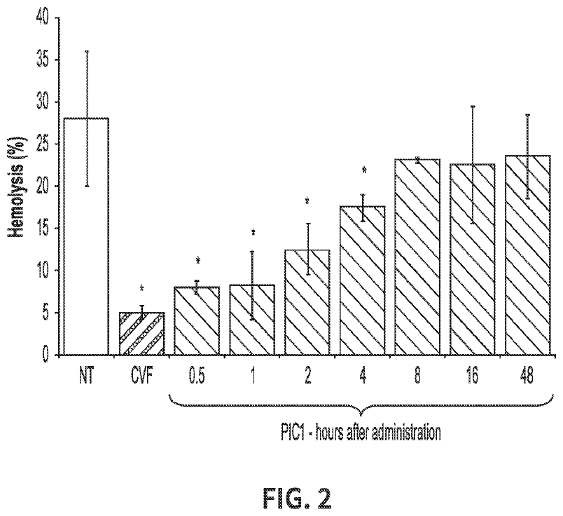

PIC1 can bind to the collagen-like region of the initiator molecule of the classical pathway, C1q, which can block activation of the associated serine proteases (C1s-C1r-C1r-C1s), and decrease generation of downstream complement effector molecules such as C3a and C5a [16]. PIC1 does not inhibit the critical immune surveillance functions of the alternative pathway [33]. Thus, PIC1 can provide for significant but temporary pharmacological inhibition of complement activity (FIG. 2), which can provide long-lasting beneficial effects as demonstrated by complex neurobehavioral testing.

Experimental data provided herein shows that animals treated with PIC1 have improvement in functional outcomes that is more consistent than that obtained with HT. PIC1 may provide for more appreciable improvement in spatial learning and memory than provided by HT. The observed variability in functional outcomes after HT is consistent with published data in animals and humans [34, 35].

Administration of PIC1 to the subject may be effective to inhibit systemic complement activation in the subject by at least 40%, 45%, 50%, 55%, 60%, 65%, 70%, 75%, 80%, 85%, or 90% over a time period of 15-60 minutes, 30-90 minutes, 1 hour to 3 hours, or 2 hours to 4 hours, as compared to a similar subject to whom PIC1 was not administered. Experimental data provided herein shows that injection with PIC1 inhibited systemic complement activation by 70% at 30 minutes after i.p. injection with gradual return to baseline by 8 hours. Treatment with PIC1 may cause a significant but temporary inhibition of complement activation. In the context of HIE, such temporary inhibition of complement activation can be sufficient to substantially improve anatomical and neurobehavioral outcomes after HIE.

Administration of PIC1 to the subject may be effective to reduce brain infarct size by at least 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78% or 80%, as compared to a similar subject to whom PIC1 was not administered. Experimental data provided herein shows that treatment with PIC1 reduced brain infarct size, and systemic inflammation as measured by C1q, C3a and C5a levels when compared to NT and HT controls. PIC1 treatment significantly reduced apoptosis as measured by TUNEL staining and resulted in superior neuroprotection as demonstrated by cell count and cell size. C1q primarily localizes in the microglia, and quantification of C1q in the ipsilateral cortex and hippocampus 48 hours after initial insult shows significant reduction in C1q expression in PIC1 animals, when compared to NT and HT controls. PIC1-treated animals showed decreased evidence of local inflammation as demonstrated by reduced microglial numbers and size, reduced astrogliosis, and decreased oxidative stress when compared to NT animals. PIC1 treatment improved fine motor performance, spatial memory and cognition after HIE.

PIC1 may be formulated into pharmaceutical compositions or formulations with one or more pharmaceutically acceptable carriers/excipients. The pharmaceutical compositions may comprise one or more of the PIC1 compounds and variants disclosed herein. The pharmaceutical compositions may also further comprise one or more other pharmaceutically or biologically active ingredients as defined above. Accordingly, also disclosed herein is a pharmaceutical composition comprising a PIC1 compound as disclosed herein. Pharmaceutical formulations comprising PIC1 preferably meet sterility, pyrogenicity, general safety, and purity standards, as required by the offices of the Food and Drug Administration (FDA).

In various embodiments, the pharmaceutically acceptable carrier or excipient is compatible with the other ingredients of a pharmaceutical composition and not deleterious to the recipient thereof. Exemplary carriers and excipients include, but are not limited to, solvents, diluents, buffers (such as, e.g., neutral buffered saline, phosphate buffered saline, or optionally Tris-HCl, acetate or phosphate buffers), solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, stabilisers, emulsifiers, sweeteners, colorants, flavorings, aromatisers, thickeners, agents for achieving a depot effect, coatings, antifungal agents, preservatives (such as, e.g., Thimerosal™, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like.

For example, the pharmaceutical composition may be formulated for parenteral administration. For example, the composition may be formulated for administration by one or more of the following means: intravenous, intracerebral, intracerebroventricular, intramuscular, or subcutaneous injection, or intravenous infusion. The parenterally acceptable aqueous solution may be pyrogen-free and have suitable pH, isotonicity and stability.

For subcutaneous or intravenous administration, the PIC1 compound(s) described herein, if desired with the substances customary therefore such as solubilizers, emulsifiers, or further auxiliaries, are brought into solution, suspension, or emulsion. The active ingredient(s) can also be lyophilized and the lyophilizates obtained used, for example, for the production of injection or infusion preparations. Suitable solvents are, for example, water, physiological saline solution, or alcohols (e.g., ethanol, propanol, glycerol), sugar solutions such as glucose or mannitol solutions, or alternatively mixtures of the various solvents mentioned. The injectable solutions or suspensions may be formulated using suitable non-toxic, parenterally-acceptable diluents, or solvents, such as mannitol, 1,3-butanediol, water, Ringer's solution, or isotonic sodium chloride solution, or suitable dispersing or wetting and suspending agents, such as sterile, bland, fixed oils, including synthetic mono- or diglycerides, and fatty acids, including oleic acid.

Alternatively, the pharmaceutical composition may be formulated for oral administration, such as for follow up dosing of PIC1. Exemplary oral administration dosage forms include, but are not limited to, pills, tablets, lacquered tablets, sugar-coated tablets, granules, hard and soft gelatin capsules, aqueous, alcoholic or oily solutions, syrups, emulsions or suspensions, or rectally, for example in the form of suppositories, percutaneous or topically (including ocular administration), for example in the form of ointments, tinctures, sprays or transdermal therapeutic systems (such as, e.g. a skin patch), or by inhalation in the form of nasal sprays or aerosol mixtures, or, for example, in the form of microcapsules, implants or rods.

Oral administration of a pharmaceutical composition comprising at least one PIC1 compound as disclosed herein, may be performed by uniformly and intimately blending together a suitable amount of said component in the form of a powder, optionally also including a finely divided solid carrier, and encapsulating the blend in, for example, a hard gelatin capsule. The solid carrier can include one or more substances, which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

The pharmaceutical compositions may be formulated so as to provide rapid, sustained, or delayed release of the active ingredient(s) contained therein, for example using micelles, liposomes or hydrophilic polymeric matrices based on natural gels or synthetic polymers.

The dosage or amount of the PIC1 compound as disclosed herein used, optionally in combination with one or more other pharmaceutically or biologically active ingredients as defined above, depends on the individual case and is, as is customary, to be adapted to the individual circumstances to achieve an optimum effect. A substantially larger dose of PIC1 may be administered to a newborn infant suffering from HIE (e.g., by parenteral administration), followed by one or more substantially smaller doses of PIC1 administered parenterally, rectally or orally.

In various embodiments, the classical complement pathway inhibitor is administered in combination with therapeutic hypothermia. PIC1 and PIC1 variants, for example, may reduce inflammation that could arise from therapeutic hypothermia while providing for additional protection against neuron loss.

### Examples

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

In the examples, means and standard error of the means (SEMs) were calculated from independent experiments. Statistical comparisons were made using the paired t-test and ANOVA where appropriate. Medians for behavioral data are compared using notched box-plots where the box represents the interquartile range (25-75th percentile) in which the horizontal line represents the median and the notch represents the confidence interval. If the notches of two boxes do not overlap, there is 95% confidence their medians differ. [18] Statistical analysis was performed with OpenEpi (Emory University) and SAS V9.3 (Cary, N.C.).

### Example 1: PIC1 Inhibits In-Vivo Complement Activity after Intraperitoneal Injection

Complement activity was assayed in plasma after i.p. injection of PIC1 in term-equivalent neonatal rats. At post-natal day 7 (P7; day of birth=P1), the rat brain is histologically similar to that of a 32- to 34-week gestation human fetus or newborn infant, i.e., cerebral cortical neuronal layering is complete, the germinal matrix is involuting, and white matter as yet has undergone little myelination. Hypoxic-ischemic brain damage in the 7-day-old rat pups is induced with unilateral common carotid artery ligation followed by systemic hypoxia produced by the inhalation of 8% oxygen/balance nitrogen, at constant temperature (37° C.) (Vanucci et al. 2005. Dev Neurosci, Vol. 27, pages 81-86).

PIC1 (IALILEPICCQERAA-dPEG24) was manufactured by PolyPeptide Group (San Diego, Calif.) to ≥95% purity as verified by HPLC and mass spectrometry analysis. The following primary antibodies were used for assays: Goat anti-rat C3 IgG (MP Biomedicals, Santa Ana, Calif.), Chicken anti-C3/C3a (Abcam, Cambridge, Mass.), Rabbit anti-rat C9 (generously provided by Professor Paul Morgan, Cardiff, UK), Mouse anti-C3aR (Thermo Fisher Scientific, Rockford, Ill.), Rabbit anti-C3aR (Bioss Inc, Woburn, Mass.), Mouse anti-C5aR (Hycult Biotech., Plymouth Meeting, Pa.), goat anti-human C1q (Complement Technology, Inc., Tyler, Tex.), mouse anti-NeuN (EMD Millipore, Temecula, Calif.), chicken anti-NeuN (EMD Millipore), mouse anti-GFAP (Sigma-Aldrich, St. Louis, Mo.), chicken anti-GFAP (Abcam), goat anti-Iba1 (Abcam), rabbit anti-C3aR (Bioss Inc, Woburn, Mass.), Mouse anti-C5aR (Hycult Biotech., Plymouth Meeting, Pa.), rabbit anti-MPO (Abeam), and rabbit anti-TMEM (Novus Bio, Centennial, Colo.). Secondary antibodies included goat anti-mouse horseradish peroxidase (HRP) (Sigma-Aldrich), goat anti-chicken IgY HRP (Genway Biotech, San Diego, Calif.), goat anti-rabbit HRP (Sigma-Aldrich), rabbit anti-goat HRP (Thermo Fisher Scientific, Grand Island, N.Y.), biotinylated goat anti-mouse IgG (H+L), biotinylated goat anti-chicken IgG (H+L) (Vector Laboratories, Burlingame, Calif.), biotinylated goat anti-rabbit IgG (H+L) (Bioss Inc.), donkey anti-goat IgG (H+L) Alexa Fluor (AF) 488/568, goat anti-chicken IgG (H+L) AF 488, goat anti-mouse IgG (H+L) AF 568, and goat anti-rabbit IgG (H+L) AF 405/488 (Life Technologies, Grand Island, N.Y.).

Timed pregnant Wistar rats (Hilltop Lab Animals Inc., Scottdale, Pa.) were purchased at embryonic day 19, housed individually, and allowed to deliver spontaneously. Pups were randomized on the day of birth to control for the litter effect (10/litter). At P10-12 (term-equivalent), pups were randomly assigned to 4 different treatment groups: no intervention (control), normothermia (NT), hypothermia-treated vehicle controls (HT), and treatment (PIC1). NT and HT animals were injected with the vehicle used to suspend PIC1. Experimental animals underwent unilateral carotid ligation followed by exposure to hypoxia (8% O₂/balanced nitrogen, Vannucci model) for 45 minutes. Pups in the HT group were placed in open jars in a temperature controlled chamber set to 28-30° C. to maintain a target rectal temperature of 31-32° C. for 6 hours, while pups in the control, NT, and PIC1 groups were kept in a separate chamber at 37° C. Pups in the PIC1 group were injected with 2 doses of PIC1 (160 mg/kg, i.p.), with the first dose at the time of carotid ligation, and the second dose four hours later. After intervention, pups were placed back with the dam, and harvested at different time points.

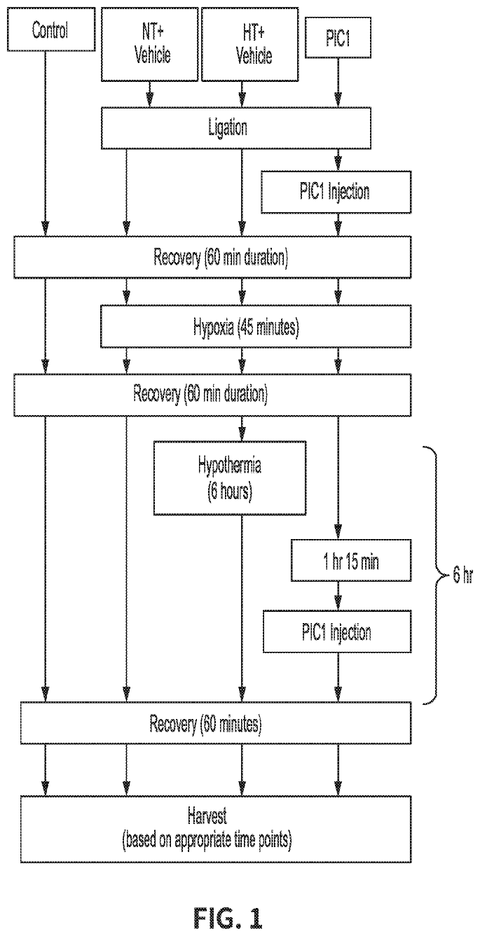

The protocol is summarized in FIG. 1. Experimental animals underwent unilateral carotid ligation followed by exposure to hypoxia (8% O₂/balanced nitrogen, Vannucci model) for 45 minutes. HT animals were placed in open jars in a temperature controlled chamber to maintain a target rectal temperature of 31 to 32° C. for 6 hours. The control, NT, and PIC1 animals were kept in a separate chamber at 37° C. PIC1 animals were injected with 2 doses of PIC1 (160 mg/kg, i.p.). The first dose of PIC1 was administered at the time of carotid ligation, and the second dose four hours later. After intervention, pups were placed back with the dam, and harvested at different time points.

Harvesting and tissue processing were performed as follows. The animals were deeply anesthetized with a fatal dose of pentobarbital (FatalPlus). After drawing blood, the animals were perfused with ice-cold PBS (followed by 10% neutral buffered formalin for perfusion fixation if being harvested for histopathology). Brains were harvested, separated into right and left hemispheres, and stored in liquid nitrogen until use. Complete mini EDTA-free protease inhibitor cocktail (Roche) was dissolved in homogenization buffer (300 mM sucrose, 0.05 mM CaCl₂), 0.1 mM MgCl₂, 0.1 mM NaHCO₃, 1 mM Na3VO4) immediately before use and each brain lobe was homogenized on ice using a mechanical homogenizer.

Whole cell protein fractions were extracted by centrifugation of the homogenate at 6000 RPM. The supernatant was collected, aliquoted and stored at −80° C. until use. Protein concentration was determined using a bicinchoninic acid assay (BCA) according to the manufacturer's recommendations (Thermo Fisher Scientific). For histopathology, 10% neutral buffered formalin was used for perfusion-fixation, brains were processed and paraffin embedded (Excalibur Pathology, Inc., Norman, Okla.) and 5 μm coronal sections were cut using a RM2125 rotary microtome (Leica Microsystems). To isolate plasma, blood was collected from cardiac puncture in EDTA tubes, incubated at room temperature for 45 minutes, then incubated on ice for 45 minutes, and centrifuged. The supernatant was collected, aliquoted, and stored at −80° C.

Cobra-venom factor (CVF) was injected i.p. into one group of animals to deplete complement activity in vivo 24 hours prior to carotid ligation [19]. On the day of surgery, a group of animals was injected with 160 mg/kg of PIC1 i.p. at the time of carotid ligation. The animals were harvested at 0.5-48 hours after injection, and plasma was collected by cardiac puncture.

Human AB erythrocytes were derived from healthy human volunteers. The erythrocytes were sensitized as described in Bonaparte et al. using rat anti-human glycophorin A IgG (LifeSpan Biosciences, Inc., Seattle, Wash.) [17]. Sensitized cells (5×10⁷), serum, and GVBS⁺⁺ (Veronal-buffered saline, 0.1% gelatin, 0.15 mM CaCl₂, and 1.0 mM MgCl) were combined in a total volume of 375 μl , and incubated for 1 hour at 37° C. in borosilicate glass tubes. The reaction was stopped with the addition of 2 ml GVBS⁻⁻ (veronal buffered saline, 0.1% gelatin, 0.01 M EDTA). The tubes were centrifuged and the absorbance of the supernatants was measured in a spectrophotometer at 412 nm.

Injection with CVF significantly depleted systemic complement activity when compared to NT control, as expected (FIG. 2). Injection with PIC1 inhibited systemic complement activation by 70% at 30 minutes after i.p. injection, with a gradual return to baseline occurring by 8 hours after injection (P<0.05 at 0.5, 1, 2 and 4 hours after injection when compared to untreated animals). Complement activity at 8, 16 and 48 hours after injection was comparable to that of untreated controls (FIG. 2). Thus, unlike CVF, PIC1 causes a temporary but significant decrease in systemic complement activity after i.p. injection.

### Example 2: PIC1 is Neuroprotective in Neonatal HIE

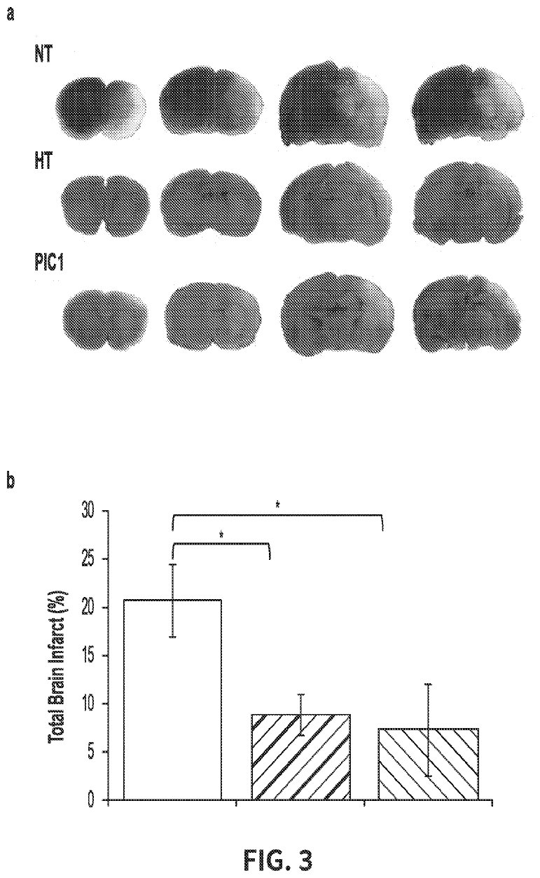

The Vannucci rat model, was used to assess whether PIC1 treatment after HIE is neuroprotective, as demonstrated by decreased brain infarct size as seen by 2,3,5-triphenyl-2H-tetrazolium chloride (TTC) staining (FIG. 3a). TTC staining was performed as follows. Harvested brains were sliced into 2 mm coronal sections, and incubated in the dark with 2% TTC at 37° C. for 30 minutes. Sections were formalin fixed before digital scanning. Image J (National Institutes of Health) was used for analysis.

Hematoxylin and eosin (H&E) and Cresyl Violet Staining were performed as follows. All washes were performed with running tap water at room temperature. Sections were deparaffinized, rehydrated, rinsed in H₂O, incubated in 20% Harris modified hematoxylin solution (8 minutes, RT), washed (5 minutes, RT), briefly dipped in 0.75% acid alcohol and washed. The washed sections were then incubated in 0.2% ammonia water solution (1 minute, RT), washed, then rinsed in 95% ethanol (10 dips), and counterstained in eosin-phloxine solution (Sigma-Aldrich) (2 minutes, RT). For Nissl staining, the sections were deparaffinized, rehydrated, rinsed in H₂O, and incubated in 0.1% cresyl violet solution (Sigma-Aldrich) (0.1 g cresyl violet acetate, 100 ml H₂O, 0.3 ml glacial acetic acid) for 10 minutes. The sections were then washed in water, dehydrated (100%, 95%, 70% ethanol, xylene), mounted (Cytoseal XYL) and cover-slipped. Whole brain section images were acquired using a generic scanner.

PIC1 animals displayed a decrease in infarct size by an average of 68% (±4.7% SEM) compared to NT animals (P=0.01) (FIG. 3b).

To assess the effect of PIC1 on cell death at 48 hours after the initial injury, TUNEL staining, alone or in combination with C1q staining, was utilized to measure DNA strand breaks that are generated during apoptosis.

TUNEL/C1q staining was performed as follows. Five (5) μm paraffin-embedded coronal brain sections on slides were incubated (1 hour; 60° C.), deparaffinized with xylene, and rehydrated with ethanol (100%, 95%, 70%; RT). Endogenous peroxidase activity was blocked (0.3% H₂O₂/MeOH; 10 minutes; RT), and heat induced antigen retrieval was performed (when indicated) using 10 mM sodium citrate, pH 6.0. 10% normal serum (from the same species as the host of the secondary antibody) was used to block and to dilute antibodies. Samples were washed twice for 10 min each, in PBST between each step.

After blocking for one hour at room temperature, tissues were incubated with 1:300 goat anti-Iba1 (Abcam), 1:250 goat anti-GFAP (Abcam), 1:500 chicken anti-NeuN (Millipore, Billerica, Mass.), or 1:100 mouse anti-NeuN (Millipore) in host appropriate blocking serum overnight at 4° C. Primary antibodies were probed with 1:500 donkey anti-goat (Abcam), goat anti-chicken (Abcam), or goat anti-mouse (Abcam) AF 488 or 568 in appropriate blocking serum for one hour at room temperature. Tissues were blocked a second time with NGS for one hour at room temperature. Samples were then probed with 1:300 mouse anti-C1q (Abcam), 1:300 rabbit anti-C9, 1:50 rabbit anti-MPO (Abcam), 1:200 rabbit anti-TMEM (Novus Bio, Centennial, Colo.), 1:100 rabbit anti-C3aR (Bioss), 1:100 Mouse anti-C5aR (Hycult), or 1:200 chicken anti-C3/C3a (Abcam) in NGS overnight at 4° C. and then with 1:500 goat anti-mouse, rabbit, or chicken (Abcam) AF 488 or 568 in NGS for one hour at room temperature.

For C1q/TUNEL co-staining, tissue was incubated with the TUNEL solution according to manufacturer's instructions (In Situ Cell Death Detection Kit, Roche Molecular Diagnostics, Pleasanton, Calif.), followed by blocking with NGS for one hour at room temperature. C1q was probed using 1:300 mouse anti-C1q (Abcam) in NGS for 30 min at 37° C., followed by incubation in 1:500 goat anti-mouse (Abcam) AF 568 for 30 min at 37° C. All samples were mounted with aqueous mounting media comprising 4′,6-diamidino-2-phenylindole (Vectashield antifade mounting medium with DAPI, Vector Laboratories, Burlingame, Calif.) and coverslipped.

Fluorescent images of the cortex were taken at 20× magnification using a digital camera (DP70, Olympus Center, Valley Forge, Pa.), mounted on a BX50 Olympus microscope. Adobe Photoshop CS5 was used to merge immunofluorescence images, and Image J (National Institutes of Health) was used for analysis.

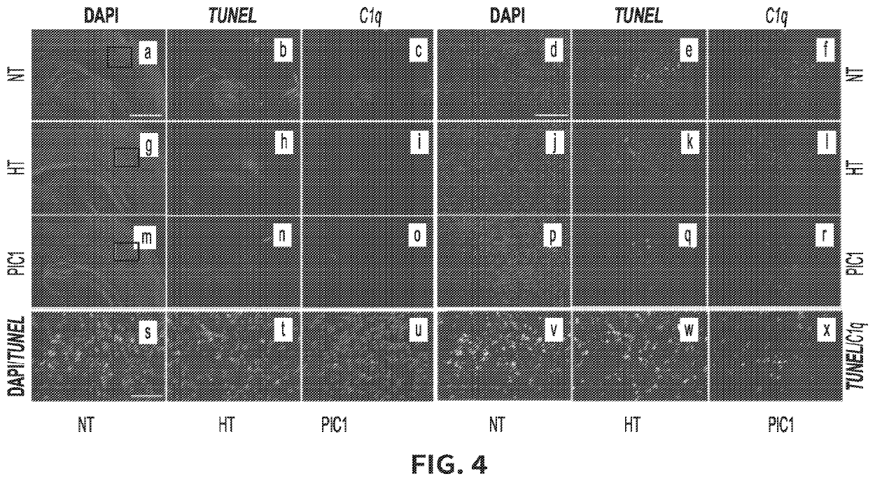

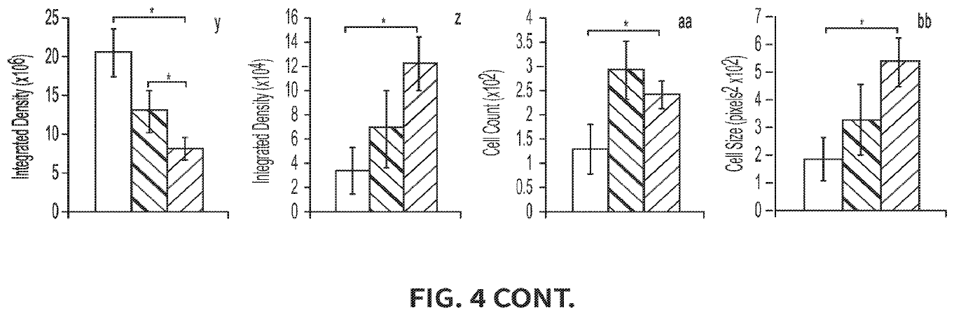

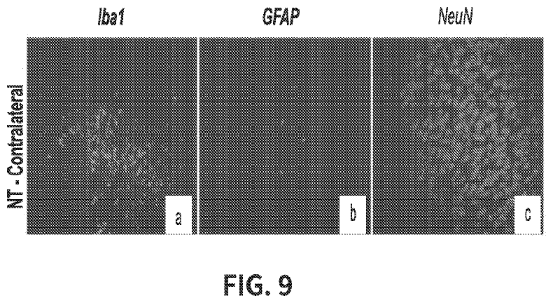

Scant TUNEL staining was seen in the normal brain and on the control contralateral non-ischemic hemisphere. A marked increase in TUNEL-positive cells was observed at 48 hours in the cortex and hippocampus of the ipsilateral hemisphere of NT animals (FIGS. 4B and 4E). Contralateral hemispheres of the brain of NT animals showed scant Iba1 and GFAP expression, and robust NeuN staining. (FIG. 9). PIC1 treatment reduced the number of TUNEL-positive cells in the ipsilateral hemisphere (FIGS. 4N and 4Q), as did treatment with HT (FIGS. 4H and 4K). Quantification of DNA fragmentation using Image J (cortex and hippocampus) showed significantly fewer TUNEL positive cells in the PIC1 group compared to NT (P=0.02) and HT (P=0.04) treated animals. (FIG. 4Y). NT animals showed markedly reduced nuclear density in the cortex as demonstrated by DAPI staining (FIGS. 4A and 4D) when compared to HT- (FIGS. 4G and 4J) and PIC1-treated (FIGS. 4M and 4P) animals. Nuclear density as demonstrated by DAPI staining was markedly improved after treatment with PIC1 when compared to NT animals (P=0.01) (FIG. 4Z). Quantification of stained nuclei also demonstrated significantly improved cell counts in PIC1-treated animals when compared to NT (P=0.03) (FIG. 4AA). While there was no significant difference in cell count of PIC1- and HT-treated animals, PIC1 animals demonstrated significantly improved nucleus size when compared to NT (P=0.02) and HT-treated animals (P=0.04) (FIG. 4BB).

NT animals also had significantly more C1q expression in the cortex and hippocampus (FIGS. 4C and 4F) when compared to HT (FIGS. 4I and 4L) and PIC1-treated animals (FIGS. 4O and 4R). Higher magnification allowed for observation of widespread DNA fragmentation (green) in nuclei in the ipsilateral cortex of NT animals (FIG. 4S). Treatment with HT (FIG. 4T) significantly reduced DNA fragmentation, as did treatment with PIC1 (FIG. 4U). See quantification in FIGS. 4Y and 4Z. There was extensive co-localization of C1q in nuclei with DNA fragmentation in NT animals (FIG. 4V), and a qualitative reduction in C1q/TUNEL co-localization in HT-treated animals (FIG. 4W) and PIC1-treated animals (FIG. 4X). Thus, PIC1 treatment significantly reduced apoptosis as measured by TUNEL staining, and provided superior neuroprotection as demonstrated by cell count and cell size when compared to NT and HT-treated animals.

### Example 3: PIC1 Decreases Local and Systemic Inflammation in Neonatal HIE

The local and systemic effects of NT, HT and PIC1 treatment on the complement cascade were measured. CNS C1q levels and systemic C1q and C5a levels were measured using a fluorescent immunoassay.

CNS C1q levels were measured as follows. 150 μg of whole cell brain lysate in 1× PBS was added to the wells of a Costar high binding, black flat bottom 96-well polystyrene microplate (Corning, Steuben County, NY) and incubated overnight at 4° C. The plates were washed 3 times with 1× PBS-0.1% Tween 20 (PBST) and blocked with 10% normal donkey serum (NDS) for one hour. A 1:50 dilution of goat anti-human C1q (Complement Technology, Inc., Tyler, Tex.) in NDS was added into the wells and incubated for one hour at room temperature (RT). After washing, a 1:500 dilution of donkey anti-goat Alexa Fluor (AF) 488 (Life Technologies, Grand Island, N.Y.) in NDS was added, and incubated for one hour at RT. Wells were then washed and blocked with 10% normal goat serum (NGS) for one hour at RT. A 1:250 dilution of mouse anti-GAPDG (Abcam, Cambridge, UK) in NGS was added into the wells and incubated for one hour at RT. Wells were washed and a 1:500 dilution of goat anti-mouse AF 568 (Life Technologies, Grand Island, N.Y.) in NGS was added to incubate for one hour at RT. The wells were washed and the plate read using a Synergy HT (BioTek, Winooski, Vt.).

Systemic C1q and C5a levels were measured as follows. 100 μl of 1:5 plasma in 1×PBS was added to the wells of a plate described above and incubated overnight at 4° C. The plates were washed 3 times with PBST and blocked with NDS or NGS for one hour. Samples were probed with either 1:50 goat anti-human C1q (Complement Technology) in NDS or 1:200 mouse anti-human C5a (Human Complement Component C5a DuoSet ELISA, R&D Systems, Inc., Minneapolis, Minn.) in NGS for one hour at RT. The wells were washed and 1:500 donkey anti-goat 488 (Life Technologies) in NDS or 1:500 goat anti-mouse AF 568 (Life Technologies) in NGS was added, and incubated for one hour at RT. The wells were washed and the plate read using a Synergy HT (BioTek). Systemic C3a levels in plasma (1:20 dilution) were measured using a standard ELISA, according to the manufacturer's instructions (Rat C3a ELISA Kit, TSZ ELISA, Waltham, Mass.).

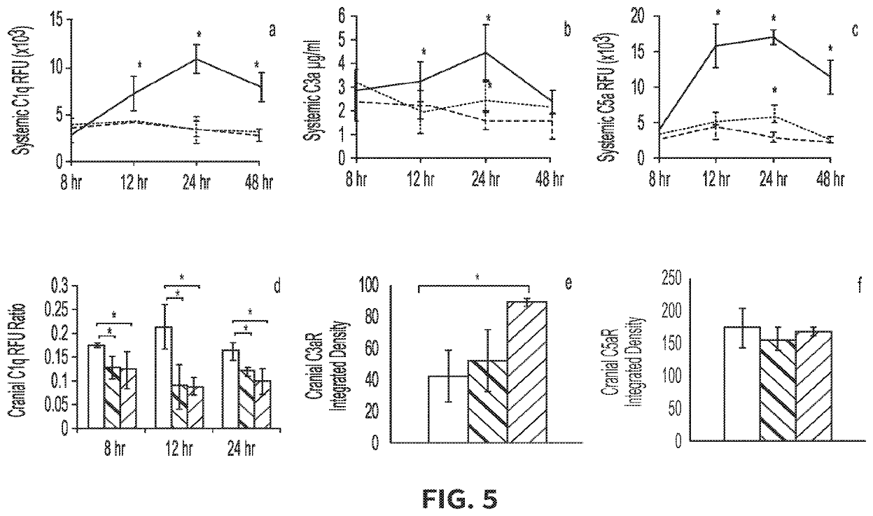

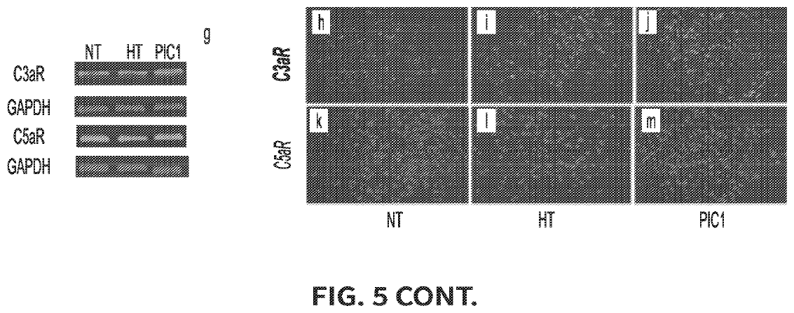

C1q levels in the plasma of NT animals were significantly higher than those of HT- and PIC1- treated animals at 8, 12 and 24 hours (P<0.05) after the initial insult, as shown in FIG. 5A. C3a levels in the plasma of NT animals were significantly higher than in plasma of HT- and PIC1-treated animals at 8, 12 and 24 hours (P<0.05) after the initial insult, as shown in FIG. 5B. Systemic C5a (FIG. 5C) levels in NT animals were significantly higher than in HT- and PIC1-treated animals at 12 and 24 (P<0.05) after the initial insult. The differences in C5a persisted out to 48 hours. Whole brain C1q levels in NT animals were significantly higher than those of HT- and PIC1-treated animals at 8, 12 and 24 hours (P<0.05) after the initial insult (FIG. 5D).

Without wishing to be bound by theory, C3a is generated at the convergence of all known complement activation pathways (i.e., the classical, alternate and lectin pathways). To investigate further, C3aR levels in the brain were measured by Western blotting and immunofluorescence (FIG. 5). Western blotting was performed as follows. 50 μg of whole cell lysate in 1× PBS was separated on a 4-20% gradient Mini-PROTEAN TGX precast gradient gel (Bio-Rad) under reducing conditions and transferred to a 0.2 μm Immun-Blot PVDF membrane (Bio-Rad, Hercules, Calif.). After washing, the membrane was blocked for 1 hour with 10% normal donkey serum (NDS) in PBS, followed by probing with goat anti-rat GAPDH (R&D Systems, Minneapolis, Minn.), followed by a 1:5000 donkey anti-goat IRDye 680 secondary antibody. The membrane was blocked with 10% normal goat sew (NGS) in PBS, followed by probing with rabbit anti-C3aR (Bioss Inc, Woburn, Mass.) or Mouse anti-C5aR (Hycult Biotech., Plymouth Meeting, Pa.), followed by a 1:5000 goat anti-rabbit IRDye 800 secondary antibody. The membrane was imaged using a LICOR Clx Odyssey imaging system using both 680 and 800 nm fluorescent channels.

The data is shown in FIGS. 5E-5M. Whole brain C3aR expression (receptor for C3a) was significantly higher in the PIC1 group as compared to NT animals (P<0.05) (FIGS. 5E, 5G-5.1). There were no significant differences in the expression of C5aR (receptor for C5a) between NT and PIC1-treated groups. (FIGS. 5F, 5G, and 5K-5M).

The consistently decreased levels of systemic C3a, C5a and C1q, and decreased deposition of C1q, C3 and C9 in the brain in HT- and PIC1-treated animals suggests that neuroprotection by these mechanisms is not dependent on C3aR levels in the brain.

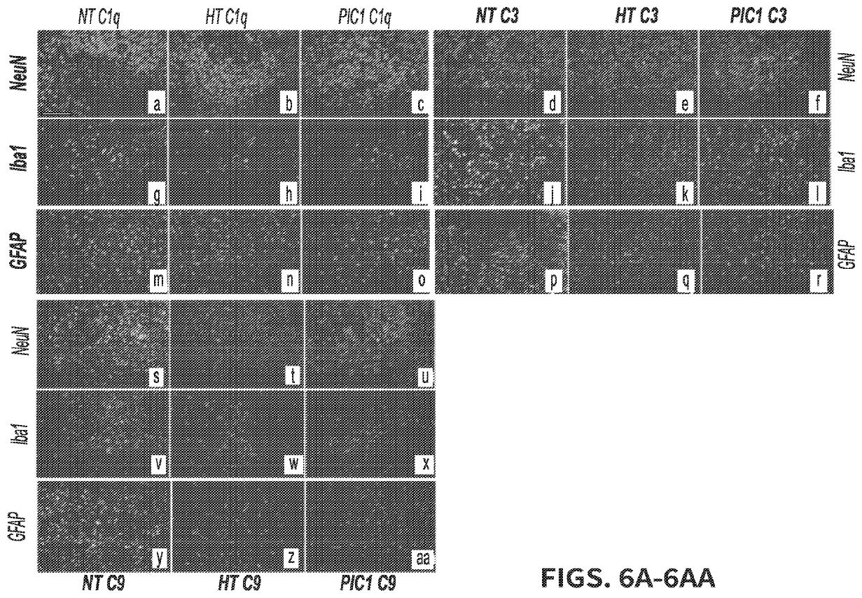

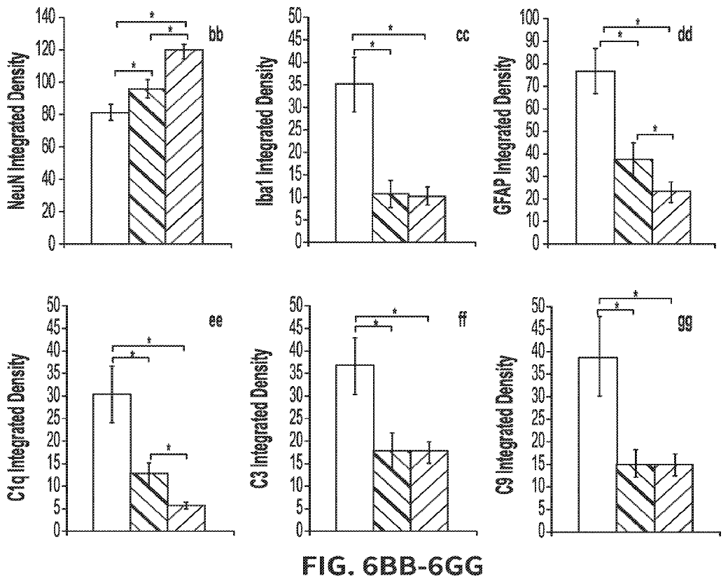

PIC1-treated animals (FIGS. 6C, 6F and 6U) demonstrated a higher neuronal density when compared to NT (FIGS. 6A, 6D, 6S) and HT-treated (FIGS. 6B, 6E and 6T) animals in the ipsilateral cortex and hippocampus (FIG. 6BB: P<0.05 NT versus PIC1 and HT versus PIC1). PIC1-treated animals showed decreased evidence of local inflammation as demonstrated by reduced microglial numbers and size (FIGS. 6I, 6L and 6X) and reduced astrogliosis (FIGS. 60, 6R and 6AA) when compared to NT animals (FIGS. 6G, 6J, 6M, 6P, 6V and 6Y) in the ipsilateral cortex and hippocampus (FIGS. 6CC and 6DD: P<0.05 NT versus PIC1).

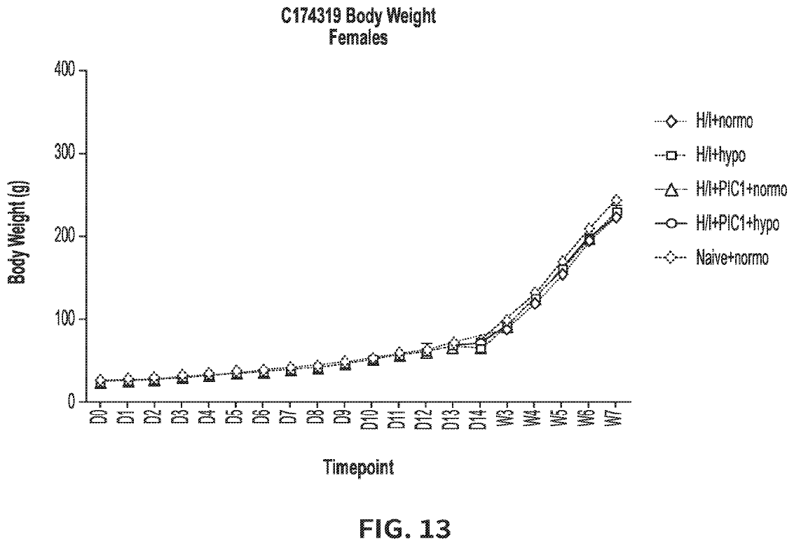

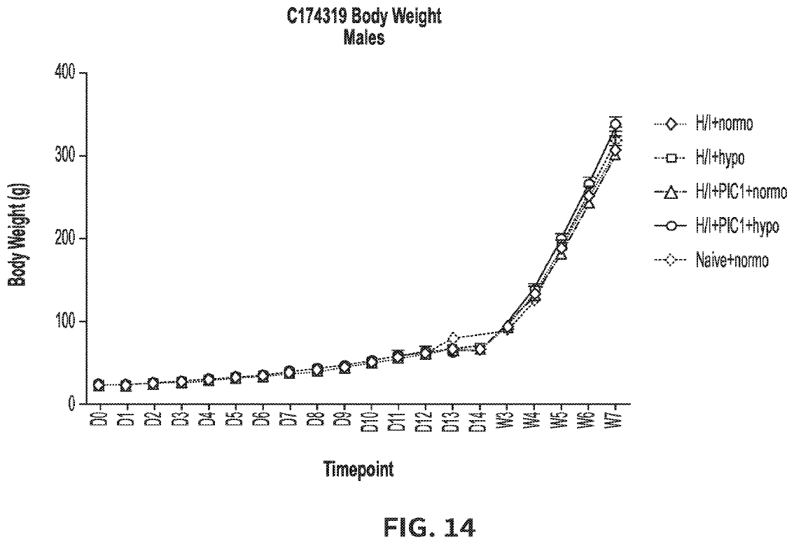

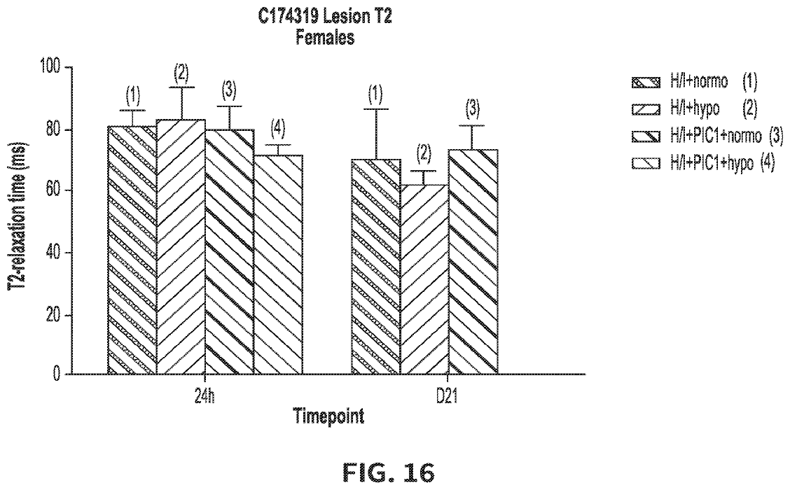

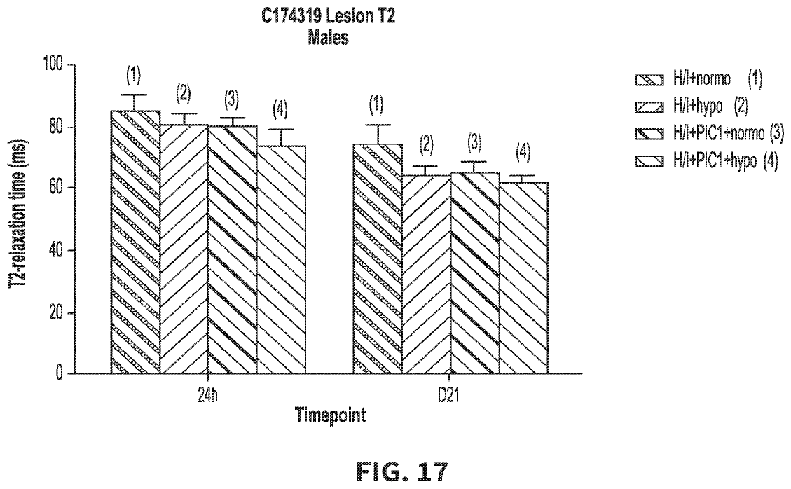

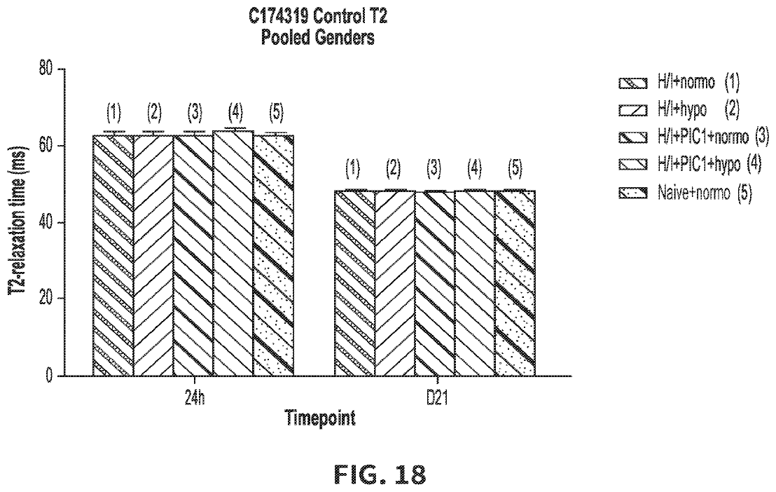

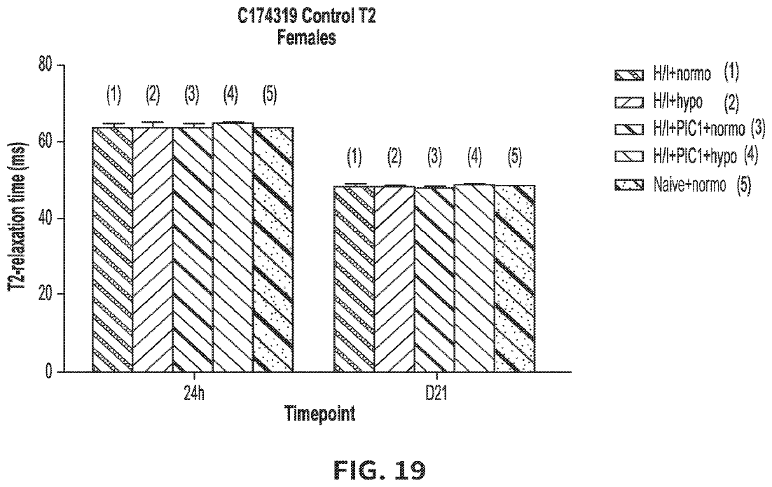

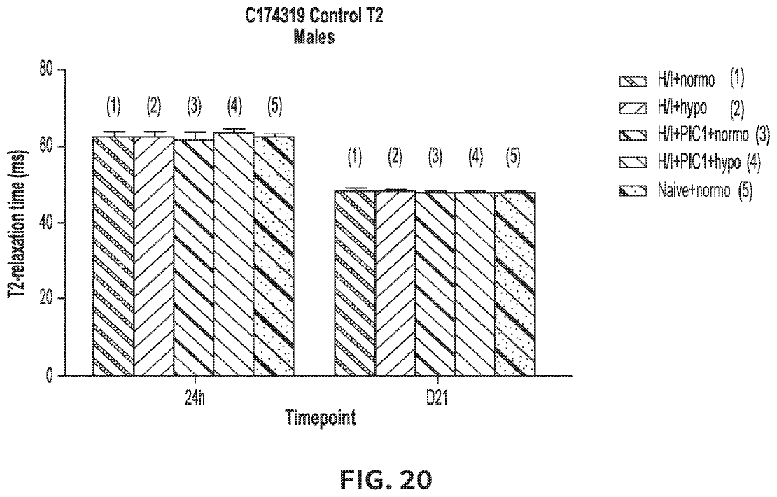

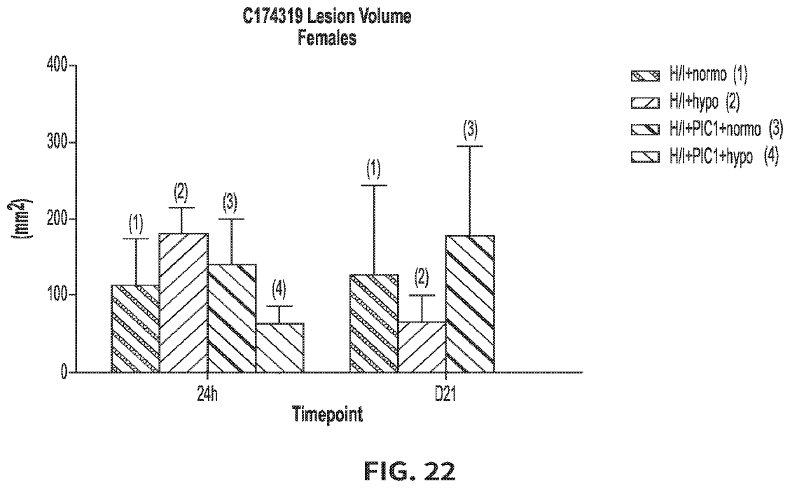

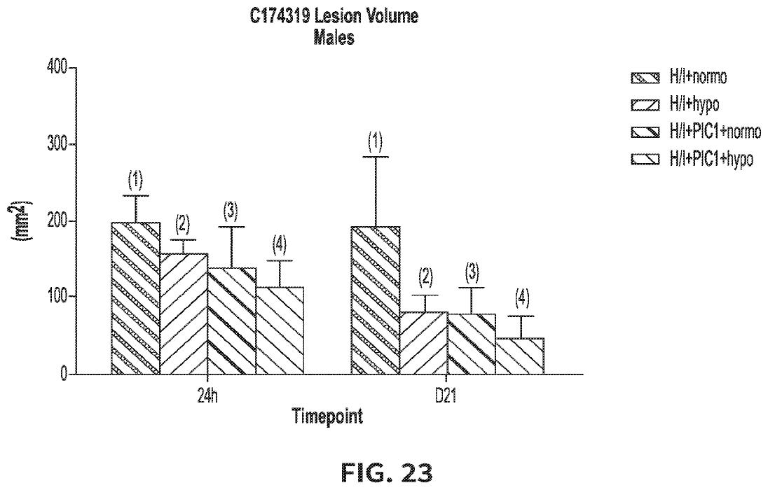

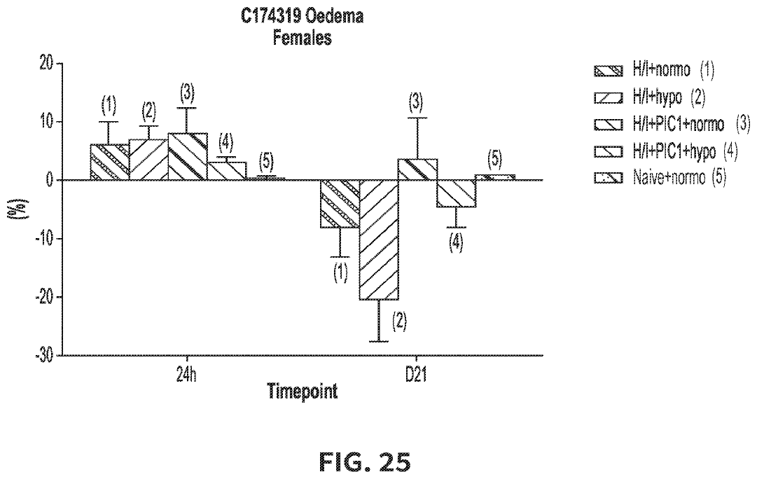

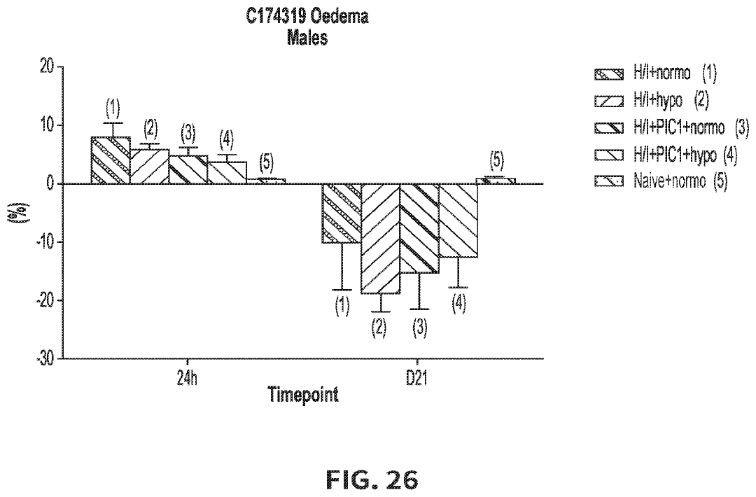

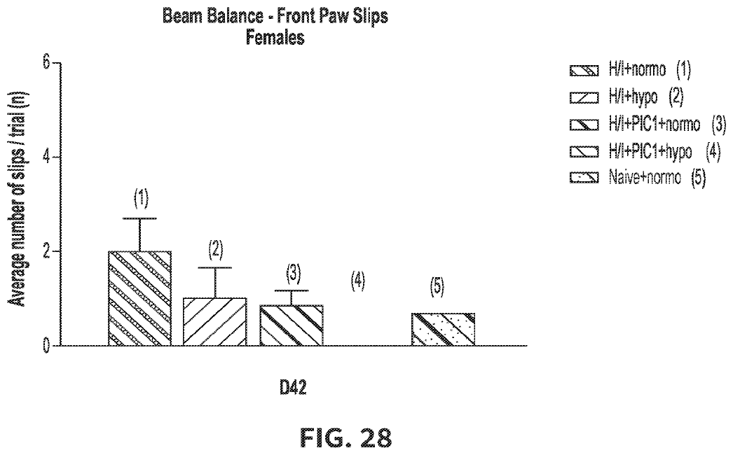

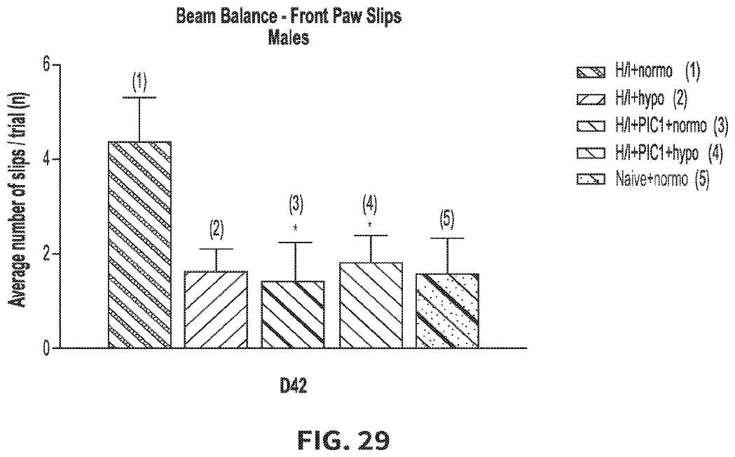

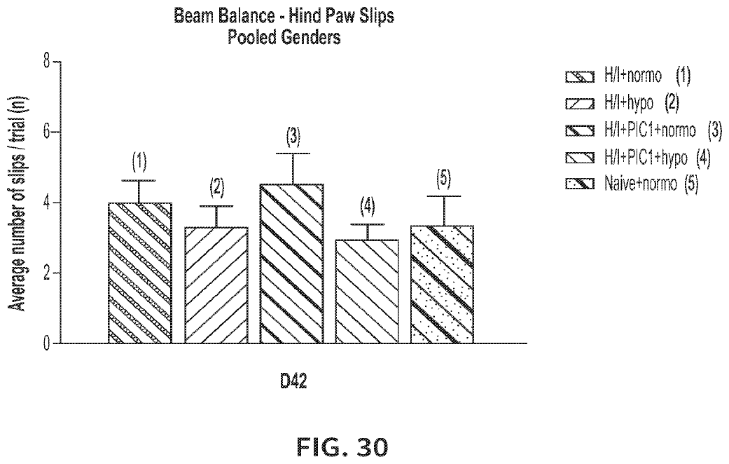

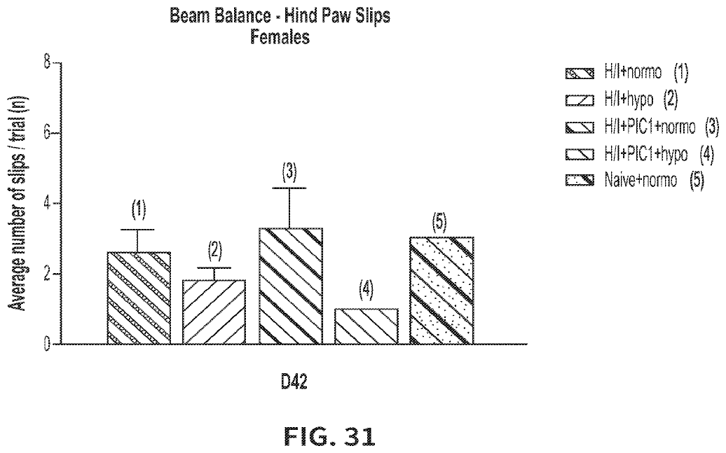

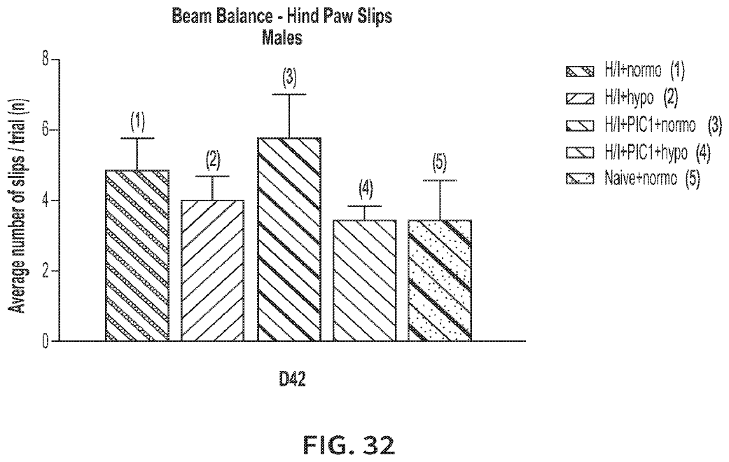

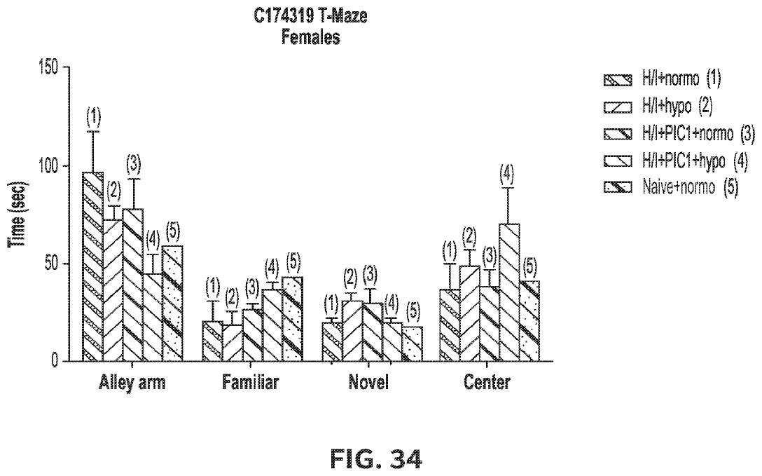

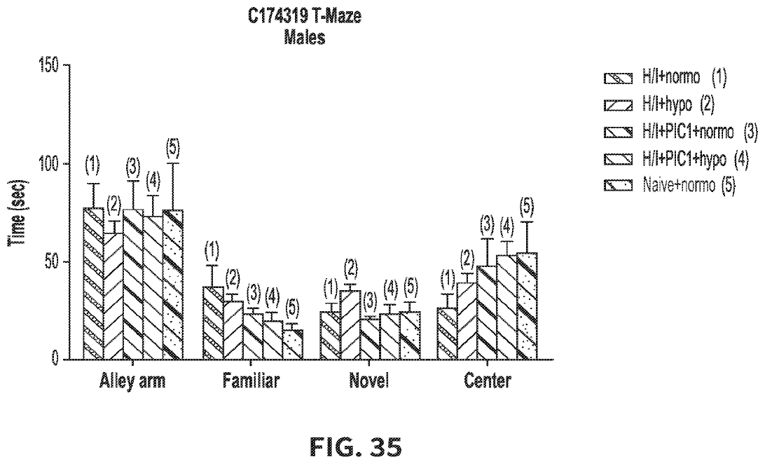

C1q primarily co-localizes in the microglia (FIGS. 6G-6I), with scant co-localization in neurons (FIGS. 6A-6C) and astrocytes (FIGS. 6M-60). C3 primarily co-localizes in the microglia (FIGS. 6J-6L) and astrocytes (FIGS. 6P-6R), with scant co-localization in neurons (FIGS. 6D-6F). C9 extensively co-localizes with neurons (FIGS. 6S-6U), microglia (FIGS. 6V-6X) and astrocytes (FIGS. 6Y-6AA).

Quantification of C1q in the ipsilateral cortex and hippocampus 48 hours after initial insult shows significant reduction in C1q expression in PIC1 animals when compared to NT and HT animals (FIG. 6EE: P<0.05 NT versus PIC1 and HT versus PIC1). Similarly, PIC1 animals showed decreased expression in C3 and C9 in the ipsilateral cortex and hippocampus 48 hours after initial insult when compared to NT animals. (FIGS. 6FF and 6GG: P<0.05 HIE versus PIC1). Thus, PIC1 treatment decreases local and systemic expression of pro-inflammatory complement effectors up to 48 hours after initial injury.

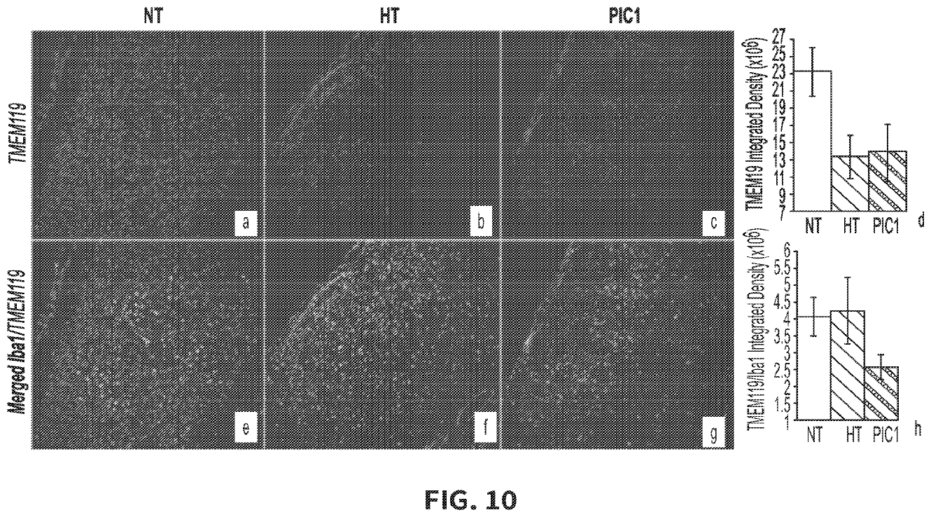

Most of the complement effectors were robustly expressed on microglia, and both PIC1 and HT brains showed significantly decreased microglial expression as measured by Iba1. Accordingly, a more specific microglial marker was used to differentiate microglia from infiltrated peripheral macrophages. TMEM119 is a reliable microglial marker that discriminates resident microglia from blood-derived macrophages in the brain [21]. HT- and PIC1-treatment resulted in an almost three-fold decrease in TMEM119 expression in the cortex, as compared to NT brains. See FIGS. 10A-10D. Interestingly, the cortex of PIC1 animals showed an almost two-fold decrease in combined Iba1/TMEM119 staining when compared to NT and HT animals. See FIGS. 10E-10H.

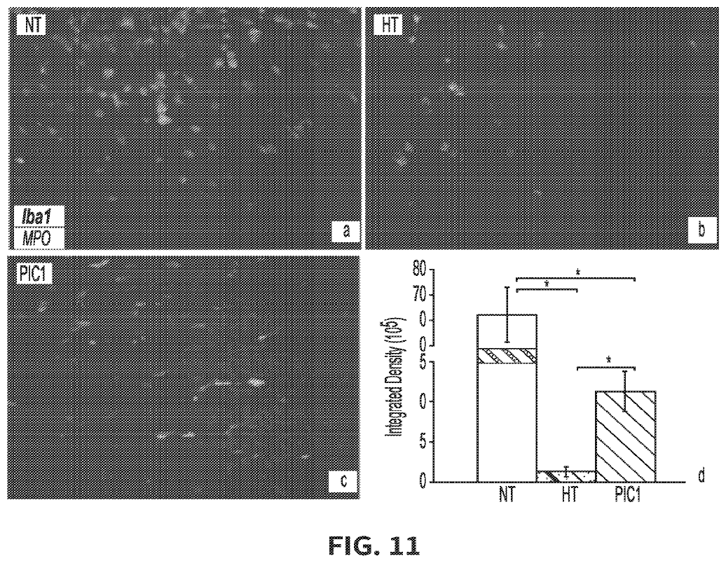

To examine the effect of PIC1 treatment on oxidant stress, expression of the enzyme myeloperoxidase (MPO) was measured in the cortex of the brain. MPO is an enzyme from neutrophils that damages invading and host cells alike. MPO is one of the markers of oxidative stress after ischemic injury and is responsible for generating reactive oxygen species (hypochlorous acid and hydrogen peroxide) [2]. The data is shown in FIG. 11. MPO expression in HT and PIC1 brains was significantly decreased (by 83%) when compared to NT animals (P<0.01). HT animals showed the greatest decrease in MPO expression. MPO extensively co-localized with microglia.

To summarize, PIC1 significantly reduced MPO production in the brain (a marker for oxidative stress), by 83% (FIG. 11). PIC1 treatment led to a more profound decrease in combined Iba1/TMEM119 staining in the brain compared with HT (FIG. 10), which could indicate that PIC1 is more effective than HT in reducing the infiltration of circulating macrophages into the brain. A combination of PIC1 treatment and hypothermia treatment could provide for both a substantial loss of MPO production, reduced resident microglia and reduced infiltration of circulating macrophages into the brain.

### Example 4: PIC1 Improves Neurocognitive Outcomes in Neonatal HIE

Long-term motor, spatial-memory and cognitive outcomes were tested in neonatal HIE after HT and PIC1 treatment, 8-12 weeks after the initial injury.

Spatial learning and memory were evaluated by measuring navigational ability of rats in a Barnes maze 8 weeks after initial injury. The Barnes maze tests memory across test sessions or trials and is more closely related to long-term memory. The test was performed using a Plexiglas maze 122 cm in diameter, with 20 equidistant holes (10.5 cm diameter) spaced every 12°, and centered 20 cm from the outer perimeter. An escape box (8 cm in depth) was placed under one of the holes. The position of the escape box was varied randomly from rat to rat, but kept constant for a given rat throughout testing. A floodlight was placed over the maze to serve as an aversive stimulus.

Prior to testing, the rats underwent habituation sessions once a day for 5 consecutive days. In each session, the rats were placed in a holding box in the middle of the maze and allowed to get acclimated for 60 seconds. The holding box was then lifted up from the rats so that the rats were allowed to explore. The test was over when either the rat found the burrowing hole and burrowed for 10 seconds or the 5-minute time limit was reached. If animals failed to reach the escape hole by the 5-minute mark, they were led to the escape hole and allowed to burrow inside for 30 seconds. On the 14th day after the habituation sessions, the rats were tested to evaluate memory retention. Latency to investigate any hole, escape latency, and number of errors during the test trials were analyzed by two-way ANOVA, followed by Bonferroni's post hoc comparison matrix. The same measurements recorded during the memory retention test were analyzed by Student's t-test. Significance was considered at p<0.05.

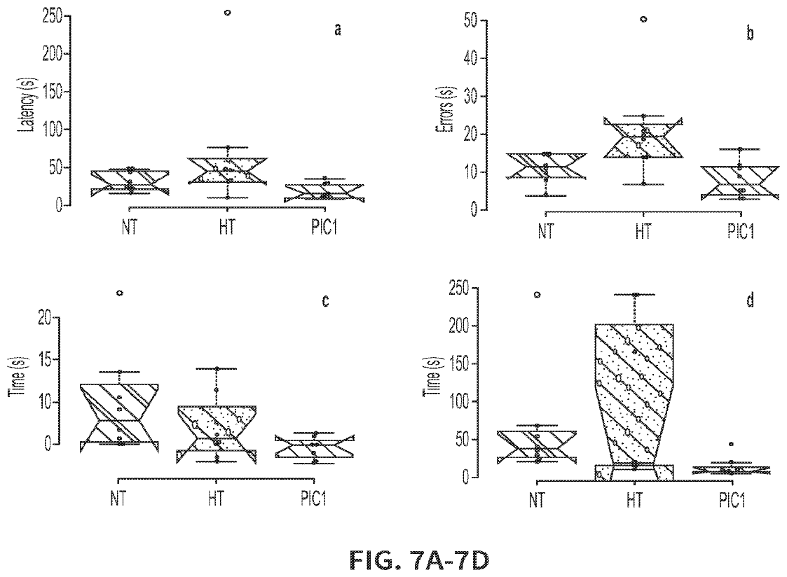

PIC1-treated animals demonstrated a significantly shorter latency in finding the escape hole, as compared with NT and HT animals (FIG. 7A). PIC1-treated animals also committed significantly fewer errors when trying to find the escape hole, as compared with HT-treated animals (FIG. 7B). Though not reaching statistical significance, PIC1-treated animals trended towards fewer errors when compared to NT animals. (P=0.06). In addition, the PIC1-treated animals were more likely to use a direct strategy to find the escape hole, rather than a random or serial strategy. Seeking patterns were classified as random when the animal randomly checks holes when trying to find the escape hole, serial when the animal uses a sequential pattern when trying to find the escape hole, or direct when the animal uses visual cues to find the escape hole.

Fine motor coordination and balance was assessed by a beam walk assay, which was performed as follows. Gross motor function and balance was evaluated using the beam walking test 6 weeks after HIE. Beams of varying widths (19 mm, 13 mm, or 6 mm) were used that led to a dark escape box. Each beam was 90 cm long and elevated 60 cm from the ground. The goal of this test is for the rat to stay upright and walk across elevated narrow beams of decreasing width (0.75 and 0.25 inches, or 19 mm and 6 mm) to a safe platform.

Rats were placed on a loading platform on the proximal end of the apparatus, and instinctively walked towards the box when placed on a beam. Rats were trained on each beam prior to the test. On the test day, rats were placed on the loading platform, and videos were recorded. Rats were given up to 5 attempts to cross each beam. Failures, i.e., inability to cross along with right and left foot slips, were analyzed by an investigator blinded to the treatment

PIC1-treated animals performed significantly better than NT animals on the 0.75 inch (FIG. 7c) and 0.25 inch (FIG. 7d) beams.

The novel object recognition test was performed at 8 weeks after HIE to test cognitive function by evaluating the differences in the exploration time of novel and familiar objects. The novel object recognition task tests for short-term, intermediate and long-term memory without the need for external motivation, reward, or punishment. Results on the test are influenced by both hippocampal and cortical injury. The test is based on the concept that when animals are exposed to a familiar and a novel object, they spend more time exploring the novel object. It is thought that the recognition of novelty requires more cognitive skills from the subject.

Rats were trained for two days prior to novel object testing. During the training sessions, the rat was allowed to explore the testing chamber, a polycarbonate box (40×40 cm) with two identical objects for three minutes. On testing day, one of the familiar objects was replaced with a novel object, made with the same material but a different shape, and allowed to explore for three minutes. Video recordings of the interactions were reviewed by blinded investigators. Object exploration was defined as the rat directing its nose toward the object at a distance of less than 2 cm. The DINOR was calculated as ((time with novel object−time with familiar object))/(total time spent with objects).

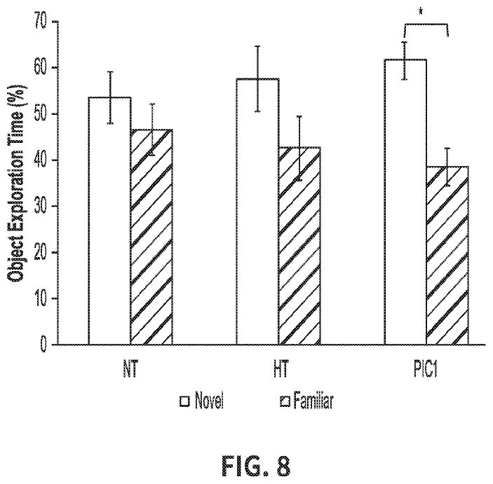

PIC1-treated animals spent significantly higher proportion of time (calculated as a percentage) exploring the novel object when compared to HT and NT animals (P<0.05) (FIG. 8). Thus PIC1 treatment improves motor, spatial memory and cognitive outcomes after HIE.

### Example 5: Assay of Brain Lesion Size, Edema and Behavior in Balance Beam and T-Maze Assays

The animal experiments in this example were carried out according to the National Institute of Health (NIH) guidelines for the care and use of laboratory animals, and approved by the National Ethics Committee (National Animal Experiment Board, Finland). The animal facility was accredited by the Association for Assessment and Accreditation of Laboratory Animal Care (AAALAC), International. Altogether 45 Wistar rat P10±1 aged pups were used for the experiment. Animals were housed in a controlled environment (temperature 21±1° C.; light cycle 13/11 h with lights on from 7 a.m. to 8 p.m. each day; relative humidity 40-70%) with ad libitum access to food and water. Pups were maintained throughout the experiments with their mothers or surrogate mothers in order to maintain normal development and care due to pre-weaning age of the pups. In the experiments, litters of individual rat mothers were mixed and balanced across the groups, to avoid any litter dependent differences in litters.

In this example is described an investigation of brain lesion size in neonatal Wistar rat pups subjected to brain hypoxia/ischemia (WI) with normothermia (36+/−1° C. rat internal temperature) and treated with PIC1.

In total, 45 Wistar rats at P10 were used in this example. Pups subjected to neonatal WI received right carotid artery ligation, with 1 h post-surgery rest. In addition, rats were subjected to 60 minutes of total hypoxia (8% O₂).

Animals were grouped as follows:

Group 1: n=10 WI operated rat pups, age of 10±1 days, subjected to 60 minutes of total hypoxia (8% O₂), normothermia for 6 hours

Group 2: n=10 WI operated rat pups, age of 10±1 days, treated with vehicle (s.c.), subjected to 60 minutes of total hypoxia (8% O₂), hypothermia for 6 hours

Group 3: n=10 H/I operated rat pups, age of 10±1 days subjected to 60 minutes of total hypoxia (8% O₂), treated with PIC1 (s.c.) (BID; dosing interval 4 hours), normothermia for 6 hours

Group 4: n=10 H/I operated rat pups, age of 10±1 days, subjected to 60 minutes of total hypoxia (8% O₂), treated with PIC1 (s.c.) (BID; dosing interval 4 hours), hypothermia for 6 hours

Group 5: n=5 No H/I operation in rat pups, age of 10±1 days, no hypoxia, normothermia for 6 hours.

Animals were treated as shown in Table 2 below.

| | | | | | | Total |
|---|---|---|---|---|---|---|
| | | TA | TA | | | doses |
| | Peptide | Dose | Dose | Dosing | Group | per |
| Group | tested | (mg/rat) | (ml/rat) | route | size | animal |
| 1 | — | — | — | — | 10 | — |
| 2 | — | — | — | — | 10 | — |
| 3 | PIC1 | 0.25 | 0.2 | s.c. | 10 | 2 |
| 4 | PIC1 | 0.25 | 0.2 | s.c. | 10 | 2 |
| 5 | — | — | — | — | 5 | — |

In Groups 3 and 4, PIC1 was administered immediately after resting phase post-hypoxia and 4 hours after the initial dose (PIC1 60 minutes post hypoxia=end of resting phase).

Neonatal hypoxia/ischemia was induced in Wistar rat pups (age of 10±1 days, P10) according to Rice et al. (1981) and with modifications. Rat pups were anesthetized with 5% isoflurane (in 70% N₂O and 30% O₂; flow 300 ml/min). During the operation the concentration of anesthetic was reduced to 1.0-1.5% to maintain adequate level of anesthesia. The rectal temperature was maintained at 37.0±1.5° C. with a homeothermic blanket system (Harvard Apparatus, UK). Just before first incision, pups received buprenorphine 0.03 mg/kg i.p. After midline skin incision, the right common carotid artery (CCA) was exposed and permanently ligated distal from the carotid bifurcation. Thereafter the wound was closed, disinfected, and the animals were allowed to recover from anesthesia for 1 hour before subjecting them to hypoxic conditions. Pups were carefully monitored for possible post-surgical complications. An additional dose of buprenorphine 0.03 mg/kg was administered s.c. twice a day during the first 48 hours post-surgery.

Therma-treatment (6 hours) was started 1 hour post-hypoxia, to maintain normothermia (36+/−1° C. rat internal temperature) or hypothermia (31° C.+/−1 rat internal temperature), as follows. After CCA ligation, suturing and recovery for 1 h, pups were placed into chamber submerged to heated (+37° C.) water bath. Chamber was ventilated with heated (gas lines submerged to +37° C. water) mixture of 8% O₂/92% N₂ while pups were subjected to hypoxia. During the hypoxia period, chamber inner temperature was monitored. Pups were kept under hypoxic conditions for 60 minutes, except in group 5. In all pups the rectal temperature was monitored to ensure normothermic body temperature during hypoxia exposure. Rectal temperature after the H/I period was measured and recorded at the end of the exposure.

PIC1 (s.c., 0.25 mg/rat, 0.2 ml/rat) was given immediately after resting phase post-hypoxia and 4 hours after the initial dose (PIC1 s.c. dosing 60 minutes post hypoxia=end of resting phase and another PIC1 s.c. dosing 300 minutes post hypoxia). For a 20 g pup the dose was 12.5 mg/kg. The test peptide PIC1 had been stored in solution in a concentration of 1.25 mg/ml at −80° C. A sufficient amount and number of stock tubes were thawed on wet ice and gently mixed by turning. No other preparation was needed. The thawed tubes were kept on ice until dosing.

Therma-treatment was started 1 hour post-hypoxia following the grouping of rats: (i) normothermia (6 hours): 36° C.+/−1 rat internal temperature, or (ii) hypothermia (6 hours): 31° C.+/−1 rat internal temperature.

After hypoxia/ischemia and infusions pups were returned to their dams and allowed to recover from anesthesia. Thereafter, the animals were observed twice a day (between 7-10 a.m. and 3-6 p.m.) during the course of the study to ensure maternal care and survival of the pups during the follow-up period. Any rat pups prematurely fulfilling the criteria for humane end-points were euthanized during the study.

When statistical analysis was performed in the experiments of this example, all values were presented as the mean±standard error of the mean. All statistical analyses were conducted with a significance level of α=0.05, using GraphPad Prism (Version 8.3, GraphPad Software, Inc., San Diego, Calif.).

Simple comparisons between two groups were performed by either unpaired Student's t-test or, where the assumption of normality were rejected by the D'Agostino-Pearson or Shapiro-Wilk test, by the Mann-Whitney U-test. Welch's t-test were used for pairwise comparisons if data were assumed to be normally distributed but with unequal variances. For comparisons between two measurements done on the same set of subjects, either paired Student's t-test or Wilcoxon signed-rank test were used as appropriate.

Comparisons involving more than three independent groups were carried out by one-way analysis of variance (ANOVA), or, if data were not normally distributed, by the Kruskal-Wallis test. If group/treatment factor were significant, post hoc multiple comparisons were performed. For comparing all means with all other means, either Tukey or Holm-Sidak tests were used. Comparisons to control group mean were done by the Dunnett's test. In case of the Kruskal-Wallis test, post hoc multiple comparisons were done by using the Dunn's test.

For repeated observations on the same group, matched values were analyzed by repeated-measures ANOVA (for normally distributed data) or the Friedman test (for groups where the assumption of normality were rejected).

Comparisons between two and more groups done at different points were analyzed by two-way repeated measures ANOVA with group/treatment as “between” factor and time as “within” factor. Main effects of group and time were initially determined and in case of their significant interaction, relevant post hoc multiple comparisons were performed.

### Body Weight Testing

The body weight of each animal was measured before the hypoxia/ischemia and daily for the first week post-H/I, followed by weekly body weight measurement until the endpoint.

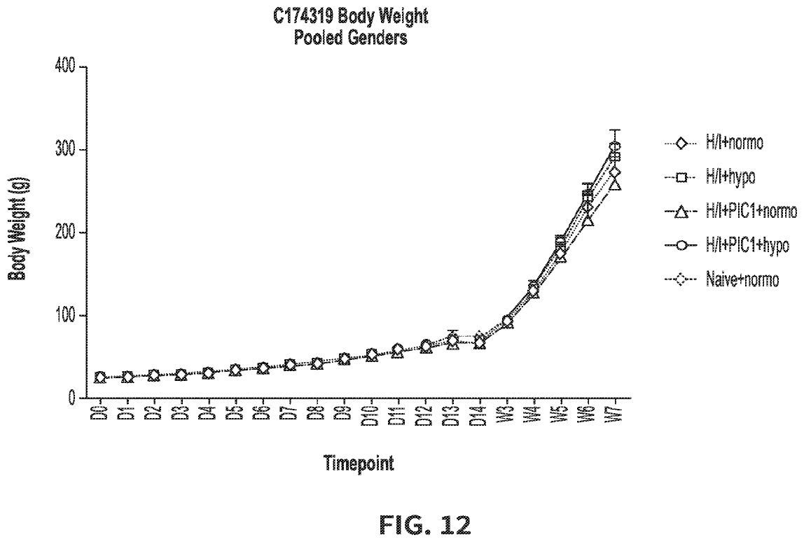

The data are shown in FIGS. 12-14. There were no significant differences in body weight in any of the groups that are comparison pooled, females and males (H/I+normo versus H/I+PIC1+normo, H/I+normo versus H/I+PIC1+hypo, H/I+hypo versus H/I+PIC1+normo, H/I+hypo versus H/I+PIC1+hypo) Two-way ANOVA (p>0.05, Two-way ANOVA, Sidak's post hoc.)

### Assay of T2 MRI for Brain Tissue Viability, Lesion Volume and Edema

MRI acquisitions were performed at 24 h after H/I to confirm the developed lesion. In addition, T2-volumetric MRI was performed 21 days after surgery for infarct volume and edema in all rats. MM was performed in a horizontal 7.0 T magnet with bore size 160 mm equipped with a gradient set capable of max. gradient strength 750 mT/m and interfaced to a Bruker Avance III console (Bruker Biospin GmbH, Ettlingen, Germany).

A volume coil (Bruker Biospin GmbH, Ettlingen, Germany) was used for transmission and a two-element surface array coil for receiving (Rapid Biomedical GmbH, Rimpar, Germany). Isoflurane-anesthetized rats (70% N2O and 30% O₂; flow 300 ml/min, induction with 5%, maintenance 1.5%) were fixed to a head holder and positioned in the magnet bore in a standard orientation relative to gradient coils.

In Mill imaging, the reduction in edema (%) was seen at 21 d in pooled genders in H/I+PIC1+normo group when compared to H/I+hypo group. The reduction in lesion T2 value was seen at day 21 in pooled genders and in female rats in H/I+PIC1+hypo group when compared to H/I+hypo group. The reduction in lesion volume was seen at 24 hours in pooled genders in H/I+PIC1+hypo group when compared to H/I+hypo group.

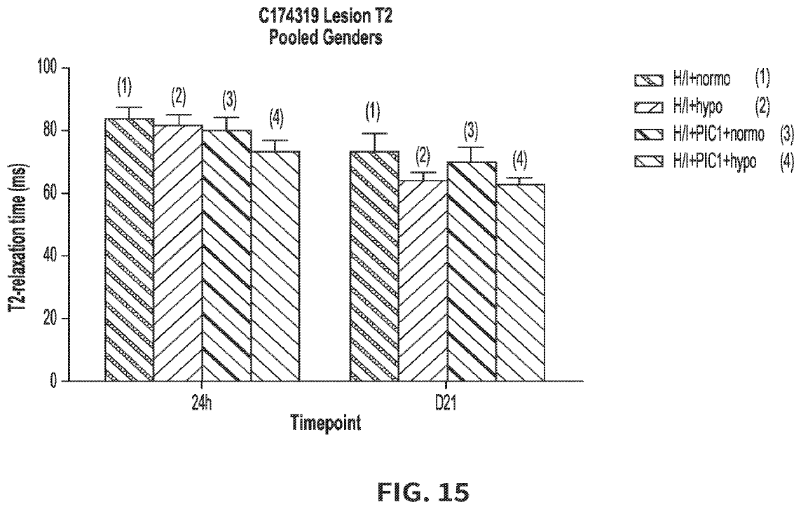

Brain tissue viability was performed as follows. The brain tissue viability of H/I operated Wistar rat pups, at the age of 10±1 days, in different groups are presented in FIGS. 15-20. Lesion T2 and Control T2 values (in milliseconds) were measured at 24 hours and 21 days after H/I, and the data are shown as average. T2-relaxation times give rise to the lesion contrast and are descriptive for tissue water environment in regards of local field dephasing effects spins experience in the tissue. In intact, healthy tissues, the dephasing effects are larger (shorter T2-values) than in lesioned tissues where the net-water is increased (or water redistributed between the water compartments).

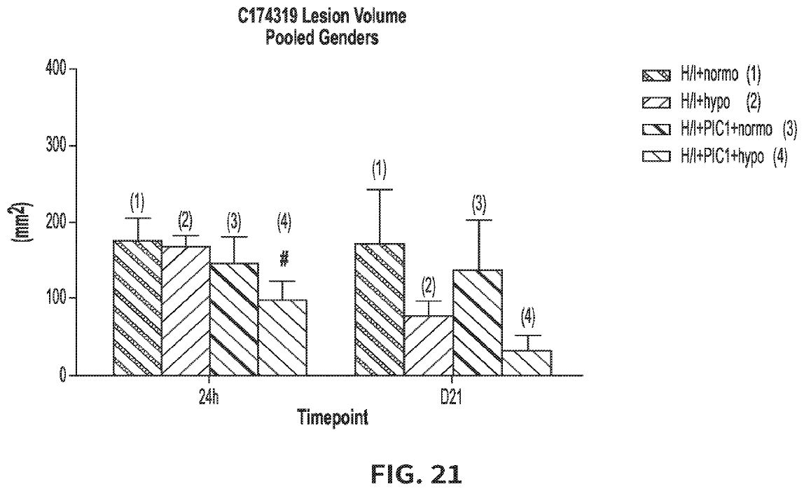

Lesion volume of H/I operated Wistar rat pups, at the age of 10±1 days, in different groups was assayed. Lesion volumes (mm3) were measured at 24 hours and 21 days after H/I. The data are presented in FIGS. 21-23, with the data are shown representing the average. According to the conducted one-way ANOVA, the reduction in lesion volume was seen at 24 hours in pooled genders in H/I+PIC1+hypo group when compared to H/I+hypo group (p<0.05, Two-way ANOVA). No other differences between study groups was seen.

According to the conducted two-way ANOVA, no changes were seen in lesion T2 value between study groups in pooled genders or when genders were separated. In addition, no changes were seen in control T2 value between study groups in pooled genders or when genders were separated.

For the determination of lesion and edema volumes, absolute T2 maps were acquired with multi-slice multi-echo sequence with following parameters; TR=2500 ms, TE=10-120 ms in 10 ms steps, matrix of 256×128, FOV of 20×20 mm², 4 transitions and 18 coronal slices of thickness 0.7 mm. For the evaluation of edema, volumes of contralateral hemisphere, ipsilateral healthy tissue and lesion were determined and the edema volume was given as subtraction of contralateral volume from ipsilateral hemisphere. Manual region of interest analysis for volumes was performed using Matlab software (MathWorks Inc., Natick, Mass.) with observer blinded to the treatment groups. T2-relaxation times (in milliseconds) give rise to the lesion contrast and are descriptive for tissue water environment in regards of local field dephasing effects spins experience in the tissue. In intact healthy tissues, the dephasing effects are larger (shorter T2-values) than in lesioned tissues where the net water is increased (or water re-distributed between the water compartments). Control values for absolute T2 were extracted from the contralateral cortex.

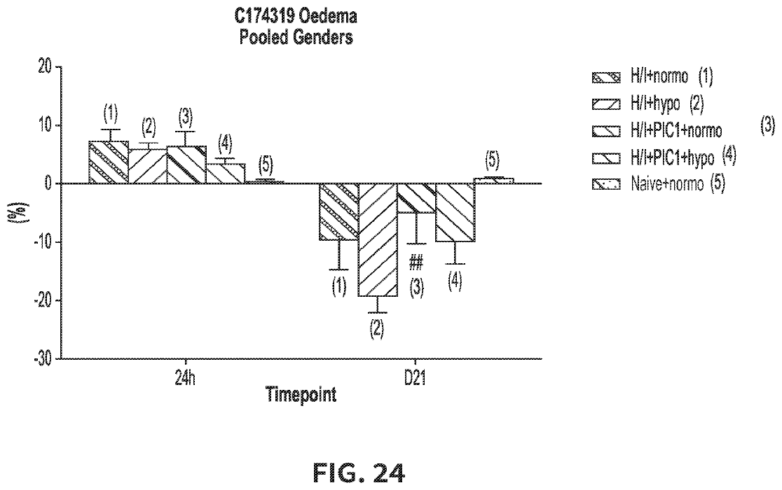

The edema percentage of H/I operated Wistar rat pups, at the age of 10±1 days, in different groups presented in FIGS. 24-26. Edema (%) were measured at 24 hours and 21 days after H/I, and the data are shown as average. Edema was calculated comparing the ipsilateral hemisphere volume to the contralateral hemisphere; in acute phase, brain swelling results positive edema values whereas in D21 scans edema is negative. This is a results from the fact that contralateral hemisphere continues normal growth from early age but ipsilateral side with lesion does not grow normally.

According to the conducted Two-way ANOVA, the reduction in edema (%) was seen at 21 days in pooled genders in H/I+PIC1+normo group when compared to H/I+hypo group (p<0.01, Two-way ANOVA). No other differences between study groups was seen.

In conclusion, PIC1 treatment combined with normothermia could reduce the edema percentage (%) in MM at day 21 after H/I in Wistar rat pups.

### Beam Balance Testing

Six weeks after surgery, the sensory motor functions of forelimbs and hindlimbs were tested using tapered/ledged beam (Zhao et al., Behav. Brain Res. 156 (2005), 85-94). The shape of the beam was square. The rats were pre-trained for 3 days to traverse the beam. The beam-walking apparatus consists of a horizontal 160 cm tapered (square) beam with underhanging ledges on each side to permit foot faults without falling. The end of the beam was connected to a black box (20.5 cm×25 cm×25 cm) with a platform at the starting point. A bright light was placed above the start point to motivate the rats to traverse the beam. The rats' performance was videotaped and were analyzed by calculating the slip ratio of the impaired (contralateral to lesion) forelimb and hindlimb (number of slips/number of total steps). Steps onto the ledge were scored as a full slip and a half slip was given if the limb touched the side of the beam. Also, the walking/running time on the beam were measured. The mean of three trials were used for statistical analyses. Analysis was carried out by an observer blind to the experimental groups.

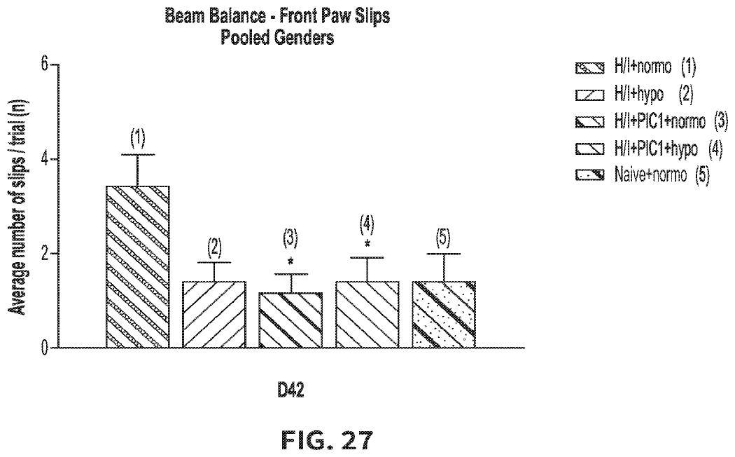

The data is shown in FIGS. 27-32. In the beam balance test, PIC1 treated rats in pooled genders and in males had fewer front paw slips when compared to H/I+normo groups six weeks after surgery. No differences were seen in hind paw slips between study groups.

### T Maze Testing

The behavior of rats in the T-maze was then assayed six weeks after ischemia surgery.

The T-maze test apparatus was plastic with two 45 cm long arms, which extend at right-angles from a 57 cm long alley (see www.noldus.com/animal-behavior-research/t-maze). The arms have a width of 10 cm and were surrounded by 10 cm high walls. The test consists of two trials at an interval of five minutes, during which time the animals are returned to their home cages. During an eight minute acquisition trial, one of the short arms was closed. In a three minute test trial, rats had access to both arms and to the alley. Time spent in each of the arms and in the long alley was assessed. Cognitively healthy rats tended to spend more time in the novel arm than in the familiar one or in the alley.

In an eight minute acquisition trial, while the novel arm is blocked, each rat was lowered to the beginning of the alley arm, facing the wall, and allowed to freely explore the maze's arms (start arm (alley arm), center and familiar arm), while the novel arm was inaccessible.

Next was a five minute interval in which the apparatus of the T-maze was cleaned with 10% Ethanol in order to remove familiar scents of the rats.

During the three minute test trial, with all three arms accessible (the wall of the novel arm was removed), each rat was allowed to freely explore the whole maze. Time spent in each of the arms was recorded using EthoVision XT 11 automated tracking system (Noldus Information Technology).

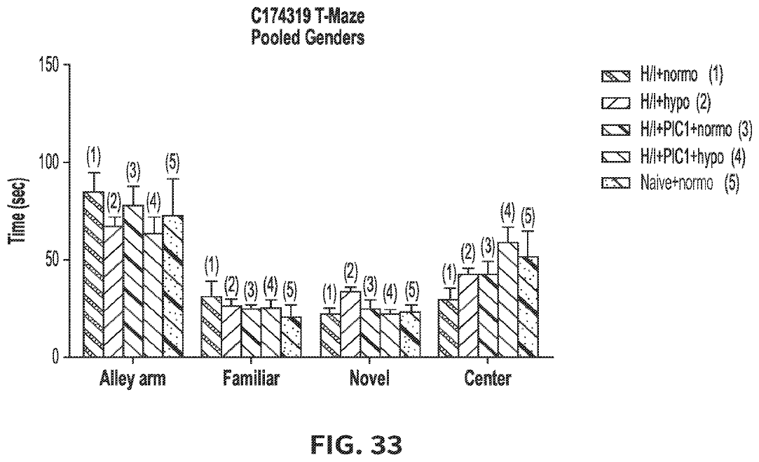

The comparisons were tested for both pooled and separated genders and no significant differences were seen. (p>0.05, Two-way ANOVA, Sidak's post hoc.) The data are shown in FIGS. 33-35.

In any of the time spent parameters studied for T-maze (alley arm, familiar arm, novel arm or center of the maze) six weeks after surgery, the rats showed no significant differences in time spent in novel and familiar arms between the study groups.

Six weeks after surgery, beam balance and T-maze tests were performed. Rats were sacrificed after all behavioral tests and no endpoint sampling was performed.

### REFERENCES

1. Arumugam T V, Magnus T, Woodruff T M, Proctor L M, Shiels I A, Taylor S M: Complement mediators in ischemia-reperfusion injury. Clin Chim Acta 2006, 374:33-45.
2. Liu F, McCullough L D: Inflammatory responses in hypoxic ischemic encephalopathy. Acta Pharmacol Sin 2013, 34:1121-1130.
3. Nauta A J, Trouw L A, Daha M R, Tijsma O, Nieuwland R, Schwaeble W J, Gingras A R, Mantovani A, Hack E C, Roos A: Direct binding of C1q to apoptotic cells and cell blebs induces complement activation. Eur J Immunol 2002, 32:1726-1736.
4. Schafer M K, Schwaeble W J, Post C, Salvati P, Calabresi M, Sim R B, Petry F, Loos M, Weihe E: Complement C1q is dramatically up-regulated in brain microglia in response to transient global cerebral ischemia. J Immunol 2000, 164:5446-5452.
5. Rocha-Ferreira E, Hristova M: Antimicrobial peptides and complement in neonatal hypoxia-ischemia induced brain damage. Front Immunol 2015, 6:56.
6. Ten V S, Sosunov S A, Mazer S P, Stark R I, Caspersen C, Sughrue M E, Botto M, Connolly E S, Pinsky D J: C1q-deficiency is neuroprotective against hypoxic-ischemic brain injury in neonatal mice. Stroke 2005, 36:2244-2250.
7. Ten V S, Yao J, Ratner V, Sosunov S, Fraser D A, Botto M, Sivasankar B, Morgan B P, Silverstein S, Stark R, et al: Complement component c1q mediates mitochondria-driven oxidative stress in neonatal hypoxic-ischemic brain injury. J Neurosci 2010, 30:2077-2087.
8. Kurinczuk J J, White-Koning M, Badawi N: Epidemiology of neonatal encephalopathy and hypoxic-ischaemic encephalopathy. Early Hum Dev 2010, 86:329-338.
9. Jacobs S E, Berg M, Hunt R, Tarnow-Mordi W O, Inder T E, Davis P G: Cooling for newborns with hypoxic ischaemic encephalopathy. Cochrane Database Syst Rev 2013, 1:Cd003311.
10. Shankaran S: Hypoxic-ischemic encephalopathy and novel strategies for neuroprotection. Clin Perinatol 2012, 39:919-929.
11. Robertson N J, Tan S, Groenendaal F, van Bel F, Juul S E, Bennet L, Derrick M, Back S A, Valdez R C, Northington F, et al: Which neuroprotective agents are ready for bench to bedside translation in the newborn infant? J Pediatr 2012, 160:544-552.e544.
12. Shankaran S, Pappas A, McDonald S A, Vohr B R, Hintz S R, Yolton K, Gustafson K E, Leach T M, Green C, Bara R, et al: Childhood outcomes after hypothermia for neonatal encephalopathy. N Engl J Med 2012, 366:2085-2092.
13. Shankaran S, Laptook A R, Pappas A, McDonald S A, Das A, Tyson J E, Poindexter B B, Schibler K, Bell E F, Heyne R J, et al: Effect of Depth and Duration of Cooling on Death or Disability at Age 18 Months Among Neonates With Hypoxic-Ischemic Encephalopathy: A Randomized Clinical Trial. Jama 2017, 318:57-67.
14. Khurshid F, Lee K S, McNamara P J, Whyte H, Mak W: Lessons learned during implementation of therapeutic hypothermia for neonatal hypoxic ischemic encephalopathy in a regional transport program in Ontario. In Paediatr Child Health. Volume 16; 2011: 153-156
15. Shah T A, Nejad J E, Pallera H K, Lattanzio F A, Farhat R, Kumar P S, Hair P S, Bass W T, Krishna N K: Therapeutic hypothermia modulates complement factor C3a and C5a levels in a rat model of hypoxic ischemic encephalopathy. Pediatr Res 2017, 81:654-662.
16. Sharp J A, Hair P S, Pallera H K, Kumar P S, Mauriello C T, Nyalwidhe J O, Phelps C A, Park D, Thielens N M, Pascal S M, et al: Peptide Inhibitor of Complement C1 (PIC1) Rapidly Inhibits Complement Activation after Intravascular Injection in Rats. PLoS One 2015, 10:e0132446.
17. Sharp J A, Whitley P H, Cunnion K M, Krishna N K: Peptide inhibitor of complement c1, a novel suppressor of classical pathway activation: mechanistic studies and clinical potential. Front Immunol 2014, 5:406.
18. Bonaparte R S, Hair P S, Banthia D, Marshall D M, Cunnion K M, Krishna N K: Human astrovirus coat protein inhibits serum complement activation via C1, the first component of the classical pathway. J Virol 2008, 82:817-827.
19. Mcgill R, Tukey J W, Larsen W A: Variations of Box Plots. http://dxdoiorg/101080/00031305197810479236 2012.
20. Kock M A, Hew B E, Bammert H, Fritzinger D C, Vogel C W: Structure and function of recombinant cobra venom factor. J Biol Chem 2004, 279:30836-30843.
21. Unger M S, Schernthaner P, Marschallinger J, Mrowetz H, Aigner L: Microglia prevent peripheral immune cell invasion and promote an anti-inflammatory environment in the brain of APP-PS1 transgenic mice. J Neuroinflammation 2018, 15:274.
22. Antunes M, Biala G: The novel object recognition memory: neurobiology, test procedure, and its modifications. In Cogn Process. Volume 13; 2012: 93-110
23. Eltzschig H K, Eckle T: Ischemia and reperfusion—from mechanism to translation. Nat Med 2011, 17:1391-1401.
24. Hassell K J, Ezzati M, Alonso-Alconada D, Hausenloy D J, Robertson N J: New horizons for newborn brain protection: enhancing endogenous neuroprotection. Arch Dis Child Fetal Neonatal Ed 2015, 100:F541-552.
25. Fonseca M I, Chu S H, Hernandez M X, Fang M J, Modarresi L, Selvan P, MacGregor G R, Tenner A J: Cell-specific deletion of C1qa identifies microglia as the dominant source of C1q in mouse brain. J Neuroinflammation 2017, 14:48.
26. Silverman S M, Kim B J, Howell G R, Miller J, John S W, Wordinger R J, Clark A F: C1q propagates microglial activation and neurodegeneration in the visual axis following retinal ischemia/reperfusion injury. Mol Neurodegener 2016, 11:24.
27. Tagin M A, Woolcott C G, Vincer M J, Whyte R K, Stinson D A: Hypothermia for neonatal hypoxic ischemic encephalopathy: an updated systematic review and meta-analysis. Arch Pediatr Adolesc Med 2012, 166:558-566.
28. Wintermark P, Boyd T, Gregas M C, Labrecque M, Hansen A: Placental pathology in asphyxiated newborns meeting the criteria for therapeutic hypothermia. Am J Obstet Gynecol 2010, 203:579.e571-579.
29. Martinello K, Hart A R, Yap S, Mitra S, Robertson N J: Management and investigation of neonatal encephalopathy: 2017 update. Arch Dis Child Fetal Neonatal Ed 2017, 102: F346-f358.
30. Stevens B, Allen N J, Vazquez L E, Howell G R, Christopherson K S, Nouri N, Micheva K D, Mehalow A K, Huberman A D, Stafford B, et al: The classical complement cascade mediates CNS synapse elimination. Cell 2007, 131:1164-1178.
31. Schafer D P, Lehrman E K, Kautzman A G, Koyama R, Mardinly A R, Yamasaki R, Ransohoff R M, Greenberg M E, Barres B A, Stevens B: Microglia sculpt postnatal neural circuits in an activity and complement-dependent manner. Neuron 2012, 74:691-705.
32. Veerhuis R, Nielsen H M, Tenner A J: Complement in the Brain. Mol Immunol 2011, 48:1592-1603.
33. Brennan F H, Anderson A J, Taylor S M, Woodruff T M, Ruitenberg M J: Complement activation in the injured central nervous system: another dual-edged sword? J Neuroinflammation 2012, 9:137.
34. Brennan F H, Gordon R, Lao H W, Biggins P J, Taylor S M, Franklin R J, Woodruff T M, Ruitenberg M J: The Complement Receptor C5aR Controls Acute Inflammation and Astrogliosis following Spinal Cord Injury. J Neurosci 2015, 35:6517-6531.
35. Gronemus J Q, Hair P S, Crawford K B, Nyalwidhe J O, Cunnion K M, Krishna N K: Potent inhibition of the classical pathway of complement by a novel C1q-binding peptide derived from the human astrovirus coat protein. Mol Immunol 2010, 48:305-313.
36. Diaz J, Abiola S, Kim N, Avaritt 0, Flock D, Yu J, Northington F J, Chavez-Valdez R: Therapeutic hypothermia provides variable protection against behavioral deficits after neonatal H I: A potential role for BDNF. Dev Neurosci 2017, 39:257-272.
37. McAdams R M, Juul S E: Neonatal Encephalopathy: Update on Therapeutic Hypothermia and Other Novel Therapeutics. Clin Perinatol 2016, 43:485-500.

The present application describes a number of examples and embodiments of the invention. Nevertheless, it must be borne in mind that various modifications of the described examples and embodiments can be developed, while not departing from the scope and the essence of the invention in principle. With this in mind, other embodiments are included in the scope of the items listed below. At that, all the numerical ranges described herein include all the sub ranges contained therein, as well as any individual values within the scope of these ranges. All publications, patents and patent applications mentioned in this description are hereby incorporated by reference.

1. A method of treating hypoxic ischemic encephalopathy (HIE) in a subject comprising administering a therapeutically effective amount of a classical complement pathway inhibitor to the subject, wherein the subject is a neonate.

2. The method of claim 1, wherein the classical complement pathway inhibitor is administered once to the subject within 24 hours of onset of the HIE.

3. The method of claim 1, wherein the classical complement pathway inhibitor is administered to the subject once a day, twice a day, three times a day, four times a day, five times a day, or six times a day for 1, 2, 3, 4, 5, 6, or 7 days after onset of the HIE.

4. A method for improving cognition and memory of a subject suffering from a neurodegenerative disease or disorder caused by neonatal hypoxic ischemic encephalopathy (HIE), the method comprising administering a therapeutically effective amount of a classical complement pathway inhibitor to the subject.

5. The method of claim 1, wherein the method is effective to inhibit C1q-mediated activation of the classical complement pathway in the brain of the subject.

6. The method of claim 1, wherein the method is effective to reduce the level of one or more of C1q, C3, C5a, or C9 in the brain of the subject.

7. The method of claim 1, wherein the method is effective to increase the expression of C3a receptor (C3aR) in the brain of the subject.

8. The method of claim 1, wherein the method is effective to reduce systemic inflammation.

9. The method of claim 1, wherein the method is effective to reduce oxidant stress in the brain of the subject.

10. The method of claim 1, wherein the method is effective to improve one or more of fine motor performance, spatial memory and cognition in the subject, as compared to another subject not treated with PIC.

11. The method of claim 1, wherein the method is effective to treat or prevent cerebral palsy.

12. The method of claim 1, wherein the classical complement pathway inhibitor is administered parenterally.

13. The method of claim 1, wherein the classical complement pathway inhibitor is administered in combination with therapeutic hypothermia.

14. The method of claim 1, wherein the classical complement pathway inhibitor is PIC1.

15. The method of claim 14, wherein the PIC1 comprises one or more PEG moieties.

16. The method of claim 14, wherein the PIC1 comprises at least about 90% sequence identity to at least one amino acid sequence selected from the group consisting of SEQ ID NO: 3-47.

17. The method of claim 14, wherein the PIC1 is PA-dPEG24.

18. The method of claim 17, wherein the PA-dPEG24 comprises the sequence of
(SEQ ID NO: 19)
IALILEPICCQERAA-dPEG24.


SEQUENCE LISTING (45 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  MOD_RES (1)..(1): Sarcosine
XALILEPICCQERAA

SEQ ID NO: 3 - protein, 15 aa, Artificial Sequence
  Description of Artificial Sequence Synthetic peptide
  MOD_RES (2)..(2): Sarcosine
IXLILEPICCQERAA

SEQ ID NO: 4 - protein, 15 aa, Artificial Sequence
  Description of Artificial Sequence Synthetic peptide
  MOD_RES (3)..(3): Sarcosine
IAXILEPICCQERAA

SEQ ID NO: 5 - protein, 15 aa, Artificial Sequence
  Description of Artificial Sequence Synthetic peptide
  MOD_RES (4)..(4): Sarcosine
IALXLEPICCQERAA

SEQ ID NO: 6 - protein, 15 aa, Artificial Sequence
  Description of Artificial Sequence Synthetic peptide
  MOD_RES (5)..(5): Sarcosine
IALIXEPICCQERAA

SEQ ID NO: 7 - protein, 15 aa, Artificial Sequence
  Description of Artificial Sequence Synthetic peptide
  MOD_RES (6)..(6): Sarcosine
IALILXPICCQERAA

SEQ ID NO: 8 - protein, 15 aa, Artificial Sequence
  Description of Artificial Sequence Synthetic peptide
  MOD_RES (7)..(7): Sarcosine
IALILEXICCQERAA

SEQ ID NO: 9 - protein, 15 aa, Artificial Sequence
  Description of Artificial Sequence Synthetic peptide
  MOD_RES (8)..(8): Sarcosine
IALILEPXCCQERAA

SEQ ID NO: 10 - protein, 15 aa, Artificial Sequence
  Description of Artificial Sequence Synthetic peptide
  MOD_RES (9)..(9): Sarcosine
IALILEPIXCQERAA

SEQ ID NO: 11 - protein, 15 aa, Artificial Sequence
  Description of Artificial Sequence Synthetic peptide
  MOD_RES (10)..(10): Sarcosine
IALILEPICXQERAA

SEQ ID NO: 12 - protein, 14 aa, Artificial Sequence
  Description of Artificial Sequence Synthetic peptide
  MOD_RES (10)..(10): Sarcosine
ALILEPICCXERAA

SEQ ID NO: 13 - protein, 15 aa, Artificial Sequence
  Description of Artificial Sequence Synthetic peptide
  MOD_RES (12)..(12): Sarcosine
IALILEPICCQXRAA

SEQ ID NO: 14 - protein, 15 aa, Artificial Sequence
  Description of Artificial Sequence Synthetic peptide
  MOD_RES (13)..(13): Sarcosine
IALILEPICCQEXAA

SEQ ID NO: 15 - protein, 15 aa, Artificial Sequence
  Description of Artificial Sequence Synthetic peptide
  MOD_RES (14)..(14): Sarcosine
IALILEPICCQERXA

SEQ ID NO: 16 - protein, 15 aa, Artificial Sequence
  Description of Artificial Sequence Synthetic peptide
  MOD_RES (15)..(15): Sarcosine
IALILEPICCQERAX

SEQ ID NO: 17 - protein, 15 aa, Artificial Sequence
  Description of Artificial Sequence Synthetic peptide
  MISC_FEATURE: N-term dPEG24
  MISC_FEATURE: C-term dPEG24
IALILEPICCQERAA

SEQ ID NO: 18 - protein, 15 aa, Artificial Sequence
  Description of Artificial Sequence Synthetic peptide
  MISC_FEATURE: N-term dPEG24
IALILEPICCQERAA

SEQ ID NO: 19 - protein, 15 aa, Artificial Sequence
  Description of Artificial Sequence Synthetic peptide
  MISC_FEATURE: C-term dPEG24
IALILEPICCQERAA

SEQ ID NO: 20 - protein, 15 aa, Artificial Sequence
  Description of Artificial Sequence Synthetic peptide
  MISC_FEATURE: C-term dPEG20
IALILEPICCQERAA

SEQ ID NO: 21 - protein, 15 aa, Artificial Sequence
  Description of Artificial Sequence Synthetic peptide
  MISC_FEATURE: C-term dPEG16
IALILEPICCQERAA

SEQ ID NO: 22 - protein, 15 aa, Artificial Sequence
  Description of Artificial Sequence Synthetic peptide
  MISC_FEATURE: C-term dPEG12
IALILEPICCQERAA

SEQ ID NO: 23 - protein, 15 aa, Artificial Sequence
  Description of Artificial Sequence Synthetic peptide
  MISC_FEATURE: C-term dPEG08
IALILEPICCQERAA

SEQ ID NO: 24 - protein, 15 aa, Artificial Sequence
  Description of Artificial Sequence Synthetic peptide
  MISC_FEATURE: C-term dPEG06
IALILEPICCQERAA

SEQ ID NO: 25 - protein, 15 aa, Artificial Sequence
  Description of Artificial Sequence Synthetic peptide
  MISC_FEATURE: C-term dPEG04
IALILEPICCQERAA

SEQ ID NO: 26 - protein, 15 aa, Artificial Sequence
  Description of Artificial Sequence Synthetic peptide
  MISC_FEATURE: C-term dPEG03
IALILEPICCQERAA

SEQ ID NO: 27 - protein, 15 aa, Artificial Sequence
  Description of Artificial Sequence Synthetic peptide
  MISC_FEATURE: C-term dPEG02
IALILEPICCQERAA

SEQ ID NO: 28 - protein, 15 aa, Artificial Sequence
  Description of Artificial Sequence Synthetic peptide
  MOD_RES (9)..(9): Sarcosine
IALILEPIXAQERAA

SEQ ID NO: 29 - protein, 14 aa, Artificial Sequence
  Description of Artificial Sequence Synthetic peptide
  MOD_RES (9)..(9): Sarcosine
IALILEPIXQERAA

SEQ ID NO: 30 - protein, 15 aa, Artificial Sequence
  Description of Artificial Sequence Synthetic peptide
  MOD_RES (7)..(7): Sarcosine
  MOD_RES (9)..(9): Sarcosine
IALILEXIXCQERAA

SEQ ID NO: 31 - protein, 15 aa, Artificial Sequence
  Description of Artificial Sequence Synthetic peptide
  MISC_FEATURE: C-term dPEG24
  MOD_RES (6)..(6): Sarcosine
IALILXPICCQERAA

SEQ ID NO: 32 - protein, 15 aa, Artificial Sequence
  Description of Artificial Sequence Synthetic peptide
  MISC_FEATURE: C-term dPEG24
  MOD_RES (11)..(11): Sarcosine
IALILEPICCXERAA

SEQ ID NO: 33 - protein, 15 aa, Artificial Sequence
  Description of Artificial Sequence Synthetic peptide
  MISC_FEATURE: C-term dPEG24
  MOD_RES (13)..(13): Sarcosine
IALILEPICCQEXAA

SEQ ID NO: 34 - protein, 15 aa, Artificial Sequence
  Description of Artificial Sequence Synthetic peptide
  MISC_FEATURE: C-term dPEG24
  MOD_RES (14)..(14): Sarcosine
IALILEPICCQERXA

SEQ ID NO: 35 - protein, 15 aa, Artificial Sequence
  Description of Artificial Sequence Synthetic peptide
  MOD_RES (6)..(7): Sarcosine
IALILXXICCQERAA

SEQ ID NO: 36 - protein, 15 aa, Artificial Sequence
  Description of Artificial Sequence Synthetic peptide
  MOD_RES (6)..(6): Sarcosine
  MOD_RES (9)..(9): Sarcosine
IALILXPIXCQERAA

SEQ ID NO: 37 - protein, 15 aa, Artificial Sequence
  Description of Artificial Sequence Synthetic peptide
  MOD_RES (7)..(7): Sarcosine
  MOD_RES (11)..(11): Sarcosine
IALILEXICCXERAA

SEQ ID NO: 38 - protein, 15 aa, Artificial Sequence
  Description of Artificial Sequence Synthetic peptide
  MOD_RES (9)..(9): Sarcosine
  MOD_RES (11)..(11): Sarcosine
IALILEPIXCXERAA

SEQ ID NO: 39 - protein, 15 aa, Artificial Sequence
  Description of Artificial Sequence Synthetic peptide
  MOD_RES (7)..(7): Sarcosine
  MOD_RES (13)..(13): Sarcosine
IALILEXICCQEXAA

SEQ ID NO: 40 - protein, 15 aa, Artificial Sequence
  Description of Artificial Sequence Synthetic peptide
  MOD_RES (9)..(9): Sarcosine
  MOD_RES (13)..(13): Sarcosine
IALILEPIXCQEXAA

SEQ ID NO: 41 - protein, 15 aa, Artificial Sequence
  Description of Artificial Sequence Synthetic peptide
  MOD_RES (7)..(7): Sarcosine
  MOD_RES (14)..(14): Sarcosine
IALILEXICCQERXA

SEQ ID NO: 42 - protein, 15 aa, Artificial Sequence
  Description of Artificial Sequence Synthetic peptide
  MOD_RES (9)..(9): Sarcosine
  MOD_RES (14)..(14): Sarcosine
IALILEPIXCQERXA

SEQ ID NO: 43 - protein, 15 aa, Artificial Sequence
  Description of Artificial Sequence Synthetic peptide
  MISC_FEATURE: C-term dPEG24
IALILAPICCQARAA

SEQ ID NO: 44 - protein, 15 aa, Artificial Sequence
  Description of Artificial Sequence Synthetic peptide
  MOD_RES (9)..(9): Sarcosine
IALILAPIXCQARAA

SEQ ID NO: 45 - protein, 15 aa, Artificial Sequence
  Description of Artificial Sequence Synthetic peptide
  MOD_RES (7)..(7): Sarcosine
IALILAXICCQARAA